(19) United States
(12) Patent Application Publication
(10) Pub. No.: US 2016/0313308 A1
(43) Pub. Date: Oct. 27, 2016

(54) DIAGNOSTIC TEST STRIP WITH SELF-ATTACHING TEST PADS AND METHODS OF USE THEREFORE

(71) Applicants: Ted Titmus, Mission Viejo, CA (US); William Pat Price, Henderson, NV (US)

(72) Inventors: Ted Titmus, Mission Viejo, CA (US); William Pat Price, Henderson, NV (US)

(21) Appl. No.: 14/694,918

(22) Filed: Apr. 23, 2015

(51) Int. Cl. G01N 33/52

(52) U.S. Cl. CPC G01N 33/525

(57) ABSTRACT

Some embodiments provide for a diagnostic test strip comprising a) a carrier strip with one or more sets of perforations; and b) one or more test pads with a plurality of legs that are attached to and extend away from the pad, wherein the sets of perforations are configured to accept the plurality of legs on each of the test pads. Other embodiments provide for a method for detecting one or more analytes in a patient sample, comprising a) contacting an embodiment of a diagnostic test strip with a patient sample so that the sample contacts the one or more test pads; and b) reading the results from the test strip.

### BACKGROUND OF THE INVENTION

1. Field of the Invention

The invention generally relates to diagnostic assay materials. More specifically, the invention relates to diagnostic test strips having one or more test pads, each of which has one or more layers, and methods for the use of said diagnostic test strips.

2. Description of the Related Art

A medical diagnostic device may be used in a variety of applications. For example, there is a continuous need for medical diagnostic devices in medical practice, research, and diagnostic procedures to conduct rapid, accurate, and qualitative or quantitative determinations of analytes which are present in biological fluids at low concentrations. However, in some circumstances the analyte of interest is present in the test sample in very small concentrations. Such circumstances require an assay to be very sensitive in order to detect the presence, absence, and/or concentration of the desired analyte. False positives and false negatives for qualitative assays can also be especially problematic.

A diagnostic test device may include a test pad containing one or more reagents for collection and analysis of biological fluids. The test pad is the portion of the diagnostic test device which is to be contacted with the test sample and through the analysis and processing of which, the existence of an analyte in the test sample can be determined. Used alone, a test pad may be delicate and susceptible to damage, such as tearing. A test pad is also susceptible to contamination from outside elements prior to use and in the process of handling and administration. Such contamination would likely alter the test results exhibited by the test pad. Thus, one method of protecting a test pad is placing it within a carrier strip in order to protect it during transport and storage prior to use and during administration of the diagnostic test. Also, once the diagnostic test has been administered, the carrier strip also acts to protect the test pad prior to processing or analysis.

Because of the delicate and sensitive nature of the test pad, the test pad is sometimes placed within the carrier strip in a manner that attempts to protect the test pad from damage and external contamination. Adhesives, such as glues, have historically been used to secure the test pad to the housing. However, using an adhesive to secure the test pad can be problematic because the use of an adhesive may actually introduce new unwanted chemical contaminants to the test pad from the adhesive itself. Chemicals and other contaminants may migrate from the adhesive securing the test pad into the test pad, thereby potentially inadvertently altering the results of the diagnostic test. Prior attempts to limit contamination from adhesives in the test pad have included using a larger test pads, which in theory necessitates that the contaminant from the adhesive travel a longer distance to actually reach and interact with the test reagent. However, using a larger test pad requires the use of increased test pad material and additional reagent, and may require excessive accumulation of the test sample in order to effectuate the test because on a larger test pad it may be more difficult for the biological sample to effectively interact with the reagent on the test pad.

What is needed is a simple, accurate assay that provides trustworthy signaling of the presence, absence, and/or concentration of one or more analytes in a given sample. It is desirable to provide a diagnostic test device having one or more test pads secured to a carrier strip such that the mechanism of securing the test pad does not contaminate the test pad or interfere with detection of analytes in a test sample. Further characteristics sought for the diagnostic test device include ease of manufacture, ease of administration, and ease of processing of the test pad. These and other objects and features of the invention will be apparent from the following description, drawings, and claims.

### SUMMARY OF THE INVENTION

Some embodiments provide for a diagnostic test strip comprising a) a carrier strip with one or more sets of perforations; and b) one or more test pads with a plurality of legs that are attached to and extend away from the pad, wherein the sets of perforations are configured to accept the plurality of legs on each of the test pads. In some embodiments, the legs of the test pads extend at least partially into the carrier strip. In other embodiments, the legs of the test pads extend through and past the opposing side of the carrier strip. In some embodiments, the legs of the test pads extend through and past the opposing side of the carrier strip and the portion of the legs that extend past the opposing side are bent to the surface of the opposing side of the carrier strip. In other embodiments, the legs of the test pads extend through and past the opposing side of the carrier strip and the portion of the legs that extend past the opposing side are bent to the surface of, and a portion of leg is further inserted into, the opposing side of the carrier strip.

Some embodiments provide for a diagnostic test strip wherein at least one of the test pads contains a test reagent. In other embodiments, there are more than one test pad and each one contains a different test reagent. In some embodiments, the test pads are substantially square. In other embodiments, the test pads are substantially circular.

Some embodiments provide for a diagnostic test strip wherein each test pad has one or more legs. In other embodiments, each test pad has at least two legs. In some embodiments, each test pad has at least three legs. In some embodiments, each test pad has four legs.

Some embodiments provide for a diagnostic test strip wherein the carrier strip is substantially porous. In other embodiments, the carrier strip is substantially non-porous. In some embodiments, the test pads are substantially porous. In other embodiments, the test pads are substantially non-porous.

Some embodiments provide for a diagnostic test strip wherein the test pads extend substantially the entire width of the carrier strip. In other embodiments, at least one test pad is substantially at one end of the carrier strip. In some embodiments, a plurality of test pads are placed sequentially over the length of the carrier strip with a defined area separating each test pad on the carrier strip.

Some embodiments provide for a diagnostic test strip wherein there are at least two or more test pads each with a different test reagent and each reagent tests for a different marker on the same analyte. In other embodiments, at least one test pad further contains a signaling reagent.

Some embodiments provide for a diagnostic test strip wherein the at least one test pad contains a reagent that tests for a saliva-borne analyte. In other embodiments, the at least one test pad contains a reagent that tests for a sputum-borne analyte. In some embodiments, the at least one test pad contains a reagent that tests for a serum-borne analyte. In other embodiments, the at least one test pad contains a reagent that tests for a plasma-borne analyte. In some embodiments, the at least one test pad contains a reagent that tests for a blood-borne analyte. In other embodiments, the at least one test pad contains a reagent that tests for a urine-borne analyte. In some embodiments, the at least one test pad contains a reagent that tests for a semen-borne analyte. In other embodiments, the at least one test pad contains a reagent that tests for an ascites-borne analyte. In some embodiments, the at least one test pad contains a reagent that tests for a cerebral spinal fluid-borne analyte.

In another embodiment, the test strip includes one or more test pads having a first transparent membrane containing a test reagent that indicates the presence of at least one reference analyte; and a second transparent membrane containing a test reagent that indicates the presence of at least one target analyte. Advantageously, each of the test reagents are arranged in a substantially single striped shape on a portion of the transparent membranes, and the transparent membranes are opposed to each other such that the striped shapes are at substantially right angles, and the at least one test pad is in fluid contact with the diagnostic test strip.

Some embodiments provide for a method for detecting one or more analytes in a patient sample, comprising a) contacting an embodiment of a diagnostic test strip with a patient sample so that the sample contacts the one or more test pads; and b) reading the results from the test strip. In some embodiments, the method further comprises contacting the test strip with one or more signaling reagents so that the one or more signaling reagents contact the one or more test pads. In some embodiments, the contacting is with a patient sample that is serum. In other embodiments, the contacting is with a patient sample that is semen. In some embodiments, the contacting is with a patient sample that is urine. In other embodiments, the contacting is directly with a patient's urine stream. In some embodiments, the contacting is with a patient sample that is saliva. In other embodiments, the contacting is with a patient's tongue. In some embodiments, the contacting is with a patient sample that is blood. In other embodiments, the contacting is directly with a source of blood. In some embodiments, the contacting is with a patient sample that is ascites. In other embodiments, the contacting is with a patient sample that is sputum. In some embodiments, the contacting is with a patient sample that is cerebral spinal fluid.

### BRIEF DESCRIPTION OF THE DRAWINGS

FIG. 1A is a top view of an embodiment of a diagnostic test strip having one test pad secured by two test pad legs.

FIG. 1B is a side view of an embodiment of a diagnostic test strip having one test pad secured by two test pad legs.

FIG. 1C is an end view of an embodiment of a diagnostic test strip having one test pad secured by two test pad legs.

FIG. 1D is a perspective view of an embodiment of a diagnostic test strip having one test pad secured by two test pad legs.

FIG. 1E is a top view of an embodiment of a diagnostic test strip having two test pads, each test pad being secured by two test pad legs.

FIG. 1F is a side view of an embodiment of a diagnostic test strip having two test pads, each test pad being secured by two test pad legs.

FIG. 1G is a perspective view of an embodiment of a diagnostic test strip having two test pads, each test pad being secured by two test pad legs.

FIG. 2A is a top view of an embodiment of a test pad having two test pad legs.

FIG. 2B is a top view of an alternative embodiment of a diagnostic test strip having one test pad secured by two test pad legs.

FIG. 2C is a side view of an alternative embodiment of a diagnostic test strip having one test pad secured by two test pad legs.

FIG. 2D is a perspective view of an alternative embodiment of a diagnostic test strip having one test pad secured by two test pad legs.

FIG. 2E is a side view of an alternative embodiment of a diagnostic test strip having two test pads, each test pad being secured by two test pad legs.

FIG. 2F is a perspective view of an alternative embodiment of a diagnostic test strip having two test pads, each test pad being secured by two test pad legs.

FIG. 3A is a top view of an embodiment of a diagnostic test strip having one test pad secured by two test pad legs and protected by two protrusions.

FIG. 3B is a side view of an embodiment of a diagnostic test strip having one test pad secured by two test pad legs and protected by two protrusions.

FIG. 3C is an end view of an embodiment of a diagnostic test strip having one test pad secured by two test pad legs and protected by two protrusions.

FIG. 3D is a perspective view of an embodiment of a diagnostic test strip having one test pad secured by two test pad legs and protected by two protrusions.

FIG. 3E is a top view of an embodiment of a diagnostic test strip having two test pads, each test pad being secured by two test pad legs and protected by two protrusions.

FIG. 3F is a side view of an embodiment of a diagnostic test strip having two test pads, each test pad being secured by two test pad legs and protected by two protrusions.

FIG. 3G is a perspective view of an embodiment of a diagnostic test strip having two test pads, each test pad being secured by two test pad legs and protected by two protrusions.

FIG. 4A is a top view of an embodiment of a diagnostic test strip having a test pad secured to the carrier strip by legs attached to the test pad.

FIGS. 4B and 4C are cross-sectional views of an embodiment of a diagnostic test strip having a test pad secured to the carrier strip by legs attached to the test pad.

FIG. 4D is a perspective view of an embodiment of a diagnostic test strip having a test pad secured to the carrier strip by legs attached to the test pad.

FIG. 5A is a top view of an embodiment of a diagnostic test strip having multiple test pads secured to the carrier strip by legs attached to the test pads.

FIG. 5B is a cross-sectional view of an embodiment of a diagnostic test strip having multiple test pads secured to the carrier strip by legs attached to the test pads.

FIG. 5C is a perspective view of an embodiment of a diagnostic test strip having multiple test pads secured to the carrier strip by legs attached to the test pads.

FIG. 6A is a top view of an embodiment of a diagnostic test strip having a test pad secured to the carrier strip by legs attached to the test pad.

FIG. 6B is a cross-sectional view of an embodiment of a diagnostic test strip having a test pad secured to the carrier strip by legs attached to the test pad.

FIG. 6C is a perspective view of an embodiment of a diagnostic test strip having a test pad secured to the carrier strip by legs attached to the test pad.

FIG. 7A is a top view of an embodiment of a diagnostic test strip having multiple test pads secured to the carrier strip by legs attached to the test pads.

FIG. 7B is a cross-sectional view of an embodiment of a diagnostic test strip having multiple test pads secured to the carrier strip by legs attached to the test pads.

FIG. 8A is a top view of an embodiment of a diagnostic test strip having shield guarding the test pad.

FIGS. 8B and 8C are cross-sectional views of an embodiment of a diagnostic test strip having shield guarding the test pad.

FIG. 8D is a perspective view of an embodiment of a diagnostic test strip having shield guarding the test pad.

### DETAILED DESCRIPTION

The present application relates to U.S. patent application Ser. No. ______, filed ______ entitled “DIAGNOSTIC TEST STRIPS WITH MULTIPLE LAMINATED LAYERS CONTAINING ONE OR MORE REAGENT-CARRYING PADS IN ONE OR MORE LAYERS”, Attorney Docket Number TTUSA.005A2, U.S. patent application Ser. No. ______, filed ______ entitled “MECHANICAL ATTACHMENT OF TEST PADS TO A DIAGNOSTIC TEST STRIP”, Attorney Docket Number TTUSA.006A2, U.S. patent application Ser. No. ______, filed ______ entitled “MECHANICAL ATTACHMENT OF TEST PADS TO A DIAGNOSTIC TEST DEVICE”, Attorney Docket Number TTUSA.007A2, U.S. patent application Ser. No. ______, filed ______ entitled “DIAGNOSTIC TEST STRIPS WITH FLASH MEMORY DEVICES AND METHODS OF USE THEREFORE”, Attorney Docket Number TTUSA.009A2, U.S. patent application Ser. No. ______, filed ______ entitled “DIAGNOSTIC TEST STRIP FOR ORAL SAMPLES AND METHOD OF USE THEREFORE”, Attorney Docket Number TTUSA.010A2, U.S. patent application Ser. No. ______, filed ______ entitled “DIAGNOSTIC TEST STRIPS HAVING ONE OR MORE TEST PAD LAYERS AND METHOD OF USE THEREFORE, Attorney Docket Number TTUSA.011A2, U.S. patent application Ser. No. ______, filed ______ entitled “SINGLE USE MEDICAL TEST PACKAGING”, Attorney Docket Number TTUSA.012A2, U.S. patent application Ser. No. ______, filed ______ entitled “DIAGNOSTIC TEST STRIPS FOR DETECTION OF PAST OR PRESENT INFECTION OF VARIOUS STRAINS OF HEPATITIS” Attorney Docket Number TTUSA.013A2, and U.S. patent application Ser. No. ______, filed ______ entitled “DIAGNOSTIC TEST STRIPS FOR DETECTION OF PRE-SPECIFIED BLOOD ALCOHOL LEVEL” Attorney Docket Number TTUSA.014A2, all of whom have the inventors Ted Titmus and William Pat Price, all of which are filed herewith this even date, all of the disclosures of which are hereby expressly incorporated by reference in their entirety and are hereby expressly made a portion of this application.

Features of the present disclosure will become more fully apparent from the following description and appended claims, taken in conjunction with the accompanying drawings. It will be understood these drawings depict only certain embodiments in accordance with the disclosure and, therefore, are not to be considered limiting of its scope; the disclosure will be described with additional specificity and detail through use of the accompanying drawings. Descriptions of unnecessary parts or elements may be omitted for clarity and conciseness, and like reference numerals refer to like elements throughout. In the drawings, the size and thickness of layers and regions may be exaggerated for clarity and convenience. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

Some embodiments of the technology disclosed herein provide for a diagnostic test device, such as a diagnostic test strip, having a test pad, and a mechanism for securing the test pad to a carrier strip. Features of the embodiments disclosed herein allow for securing the test pad to the carrier strip in a manner which prevents contamination and damage to the test pad. One or more substantially thin test pads may be utilized and may be secured to the carrier strip without the use of traditional adhesives contacting the test pad. Optionally, one or more test pads may be secured without any use of traditional adhesives. The one or more test pads may contain test reagents and/or signaling reagents that detect analytes. Test pads, test reagents, and signaling reagents are described in more detail below. Also described in more detail below, analytes may be reference analytes, or they may be target analytes.

Other embodiments provide for a method of detecting one or more analytes in a patient sample by contacting one or more test pads of an embodiment of a diagnostic test strip with a patient sample and reading the results from the embodiment. Moreover, embodiments may be directly contacted with a patient's sample or the source of the sample. These methods include contacting the test strip with one or more signaling reagents so that the one or more reagents contact the one or more test pads.

Any method's results may be read visually by an embodiment's user, if the application so desires, and/or any method's results may be stored in a memory device for recordation and later access. Alternatively, the results may be read by someone other than the user or the supplier of the sample. In some circumstances, the results of the method will be restricted from the user of the embodiment and/or the supplier of the sample analyzed.

Embodiments of the invention can be used to detect any analyte which has heretofore been assayed using known immunoassay procedures, or known to be detectable by such procedures. Furthermore, it is envisioned that known methods can be modified as needed to afford suitable test reagents and/or signaling reagents that will detect analytes that are similar to analytes that have been previously detected using known procedures.

As disclosed below, various features of the embodiments and methods of using the embodiments enable both trained and untrained personnel to reliably detect the presence, absence, and/or concentration of one or more analytes in a sample. Indeed, features of the embodiments and methods for their use allow for the detection of extremely small quantities of one or more particular analytes while avoiding false positives and false negatives. Furthermore, features of the embodiments and methods for their use allow for accurate and trustworthy attainment and/or storage of information related to the tested sample. Optionally, embodiments may both produce a signal that communicates information to the user and/or store information related to the test sample in one or more memory devices. Consequently, the invention is ideal for use in both prescription and over-the-counter assay test kits which will enable a consumer to self diagnose themselves and others, or test food and/or water prior to consumption.

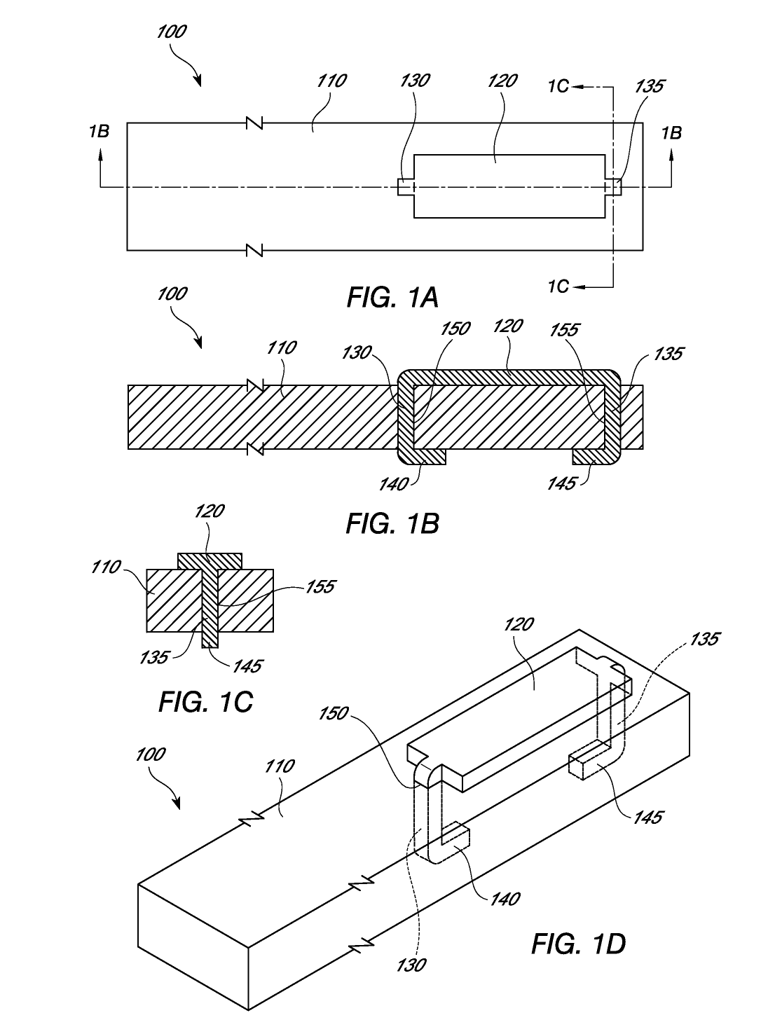

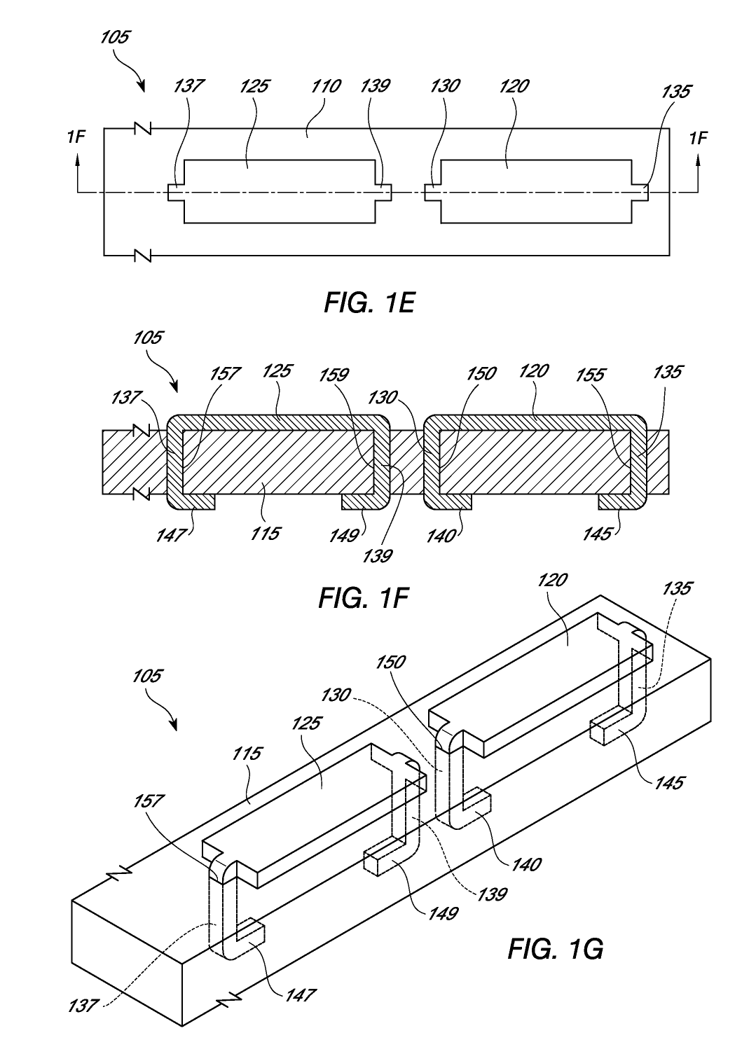

Referring to the drawings, FIGS. 1A-1D illustrate schematically an embodiment of a diagnostic test strip, 100, having a carrier strip, 110, and one test pad, 120, located on the diagnostic test strip. FIG. 1A is a top view of an embodiment of a diagnostic test strip having one test pad secured by two test pad legs, 130 and 135. In FIG. 1A, test pad legs, 130 and 135 facilitate the securing of test pad 120 to the diagnostic test strip.

FIG. 1B is a schematic illustration of a side view of diagnostic test strip 100. In FIG. 1B, carrier strip perforations 150 and 155 form holes such that they readily accept test pad legs 130 and 135, respectively. Additionally, FIG. 1B illustrates the angling of test pad legs 130 and 135 upon extension through perforations 150 and 155, respectively. As FIG. 1B illustrates, test pad legs 130 and 135 are angled upon exiting the perforations in the carrier strip opposite the side of test pad 120. Denoted as 140 and 145, respectively, the angled test pad legs further facilitate the securing of test pad 120 to the diagnostic test strip.

FIG. 1C is a schematic illustration of an end view of diagnostic test strip 100. In FIG. 1C, one can readily appreciate that test pad 120 is secured to carrier strip 110 by the acceptance of test pad leg 135 in perforation 155. Additionally, FIG. 1C illustrates the extension of test pad leg 135 through perforation 155 to afford a portion of the test pad leg 145, which is angled under the carrier strip opposite test pad 120 to facilitate the securing of the test pad to the diagnostic test strip.

FIG. 1D is a schematic illustration of perspective view of diagnostic test strip 100. In FIG. 1D, carrier strip perforations 150 and 155 form holes such that they readily accept test pad legs 130 and 135, respectively. Additionally, FIG. 1B illustrates the angling of test pad legs 130 and 135 upon extension through perforations 150 and 155, respectively. As FIG. 1B illustrates, test pad legs 130 and 135 are angled upon exiting the perforations in the carrier strip opposite the side of test pad 120. Denoted as 140 and 145, respectively, the angled test pad legs further facilitate the securing of test pad 120 to the diagnostic test strip.

Referring to the drawings, FIGS. 1E-1G illustrate schematically an embodiment of a diagnostic test strip, 105, having a carrier strip, 115, and two test pads, 120 and 125, located on the diagnostic test strip. FIG. 1E is a top view of an embodiment of a diagnostic test strip, 105, having two test pads, 120 and 125, each test pad being secured by two test pad legs, 130 and 135, and 137 and 139, respectively.

FIG. 1F is a schematic illustration of a side view of an embodiment of a diagnostic test strip, 105, having two test pads, 120 and 125, each test pad being secured by two test pad legs, 130 and 135, and 137 and 139, respectively. In FIG. 1F, carrier strip perforations 150 and 155 form holes such that they readily accept test pad legs 130 and 135, respectively. Furthermore, carrier strip perforations 157 and 159 form holes such that they readily accept test pad legs 137 and 139, respectively. Additionally, FIG. 1F illustrates the angling of test pad legs 130, 135, 137, and 139 upon extension through perforations 150 and 155, respectively, and 157 and 159, respectively. As FIG. 1F illustrates, test pad legs 130 and 135 are angled upon exiting the perforations in the carrier strip opposite the side of test pad 120. Denoted as 140 and 145, respectively, the angled test pad legs further facilitate the securing of test pad 120 to the diagnostic test strip. Furthermore, FIG. 1F illustrates that test pad legs 137 and 139 are angled upon exiting the perforations in the carrier strip opposite the side of test pad 125. Denoted as 147 and 149, respectively, the angled test pad legs further facilitate the securing of test pad 125 to the diagnostic test strip.

FIG. 1G is a schematic illustration of perspective view of diagnostic test strip 105. In FIG. 1G, carrier strip perforations 150 and 155 form holes such that they readily accept test pad legs 130 and 135, respectively. Furthermore, carrier strip perforations 157 and 159 form holes such that they readily accept test pad legs 137 and 139, respectively. Additionally, FIG. 1F illustrates the angling of test pad legs 130, 135, 137, and 139 upon extension through perforations 150 and 155, respectively, and 157 and 159, respectively. As FIG. 1F illustrates, test pad legs 130 and 135 are angled upon exiting the perforations in the carrier strip opposite the side of test pad 120. Denoted as 140 and 145, respectively, the angled test pad legs further facilitate the securing of test pad 120 to the diagnostic test strip. Furthermore, FIG. 1F illustrates that test pad legs 137 and 139 are angled upon exiting the perforations in the carrier strip opposite the side of test pad 125. Denoted as 147 and 149, respectively, the angled test pad legs further facilitate the securing of test pad 125 to the diagnostic test strip.

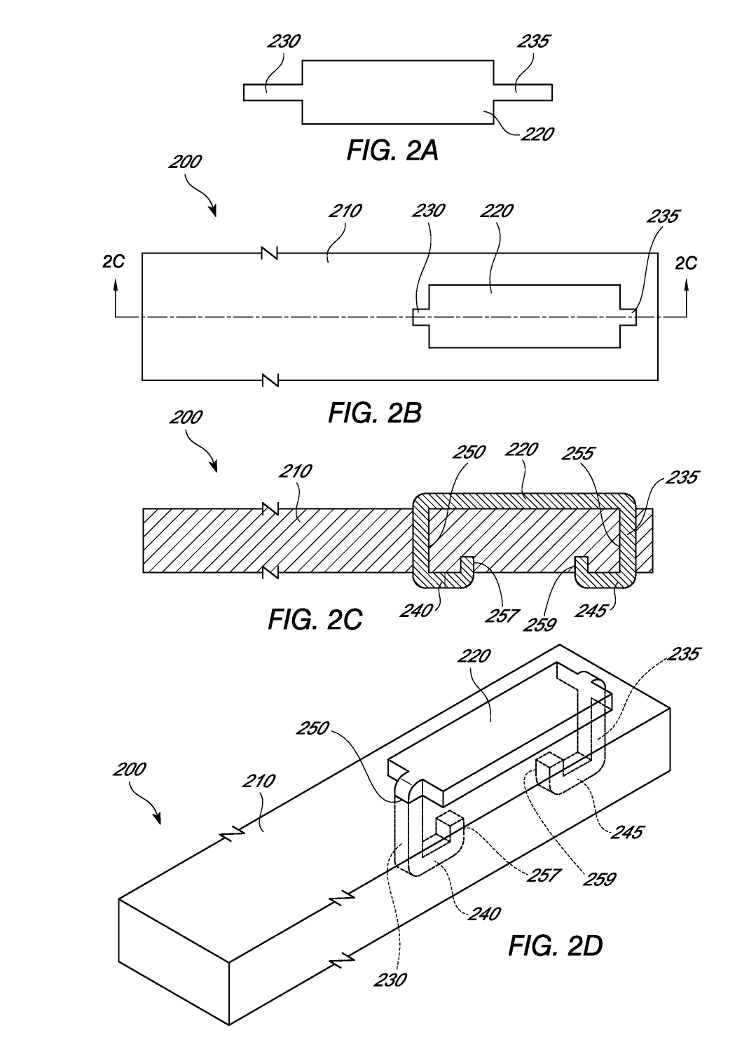

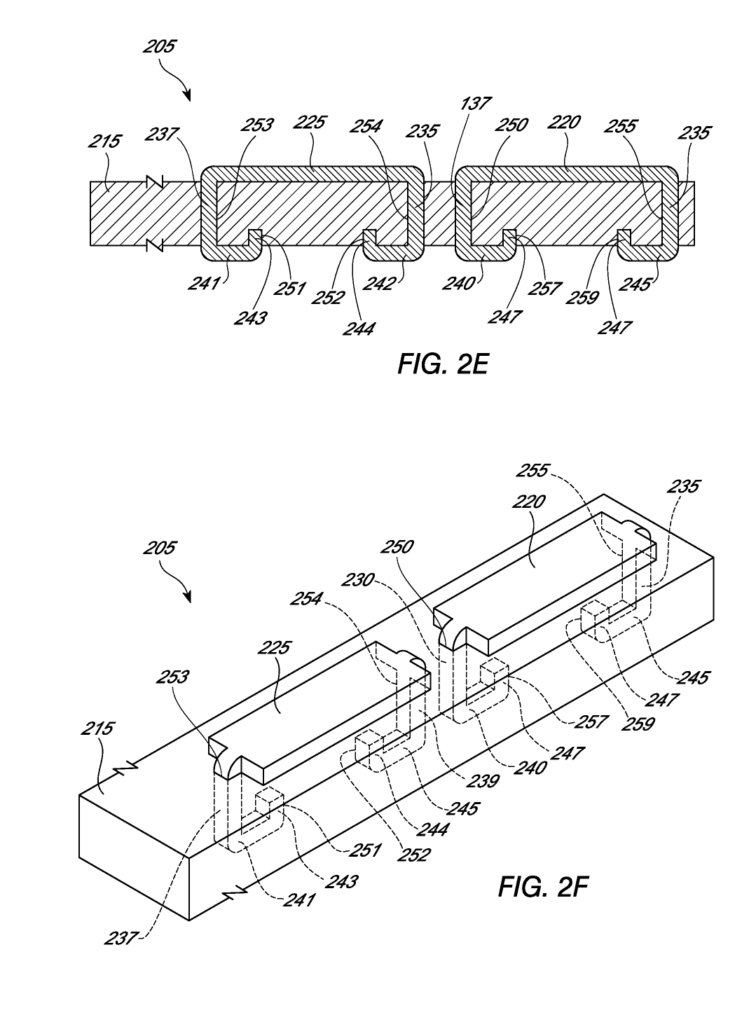

Referring to the drawings, FIGS. 2A-2D illustrate schematically an embodiment of a test pad, 220, and diagnostic test strip, 200, having a carrier strip, 210. FIG. 2A is a schematic illustration of a top view of an embodiment of a test pad, 220, having two test pad legs, 230 and 235. FIG. 2B schematically represents a top view of diagnostic test strip 200 having test pad 220 secured by two test pad legs, 230 and 235. In FIG. 2B, test pad legs 230 and 235 facilitate the securing of test pad 220 to the diagnostic test strip.

FIG. 2C is a schematic illustration of a side view of diagnostic test strip 200. In FIG. 2C, carrier strip perforations 250 and 255 form holes such that they readily accept test pad legs 230 and 235, respectively. Additionally, FIG. 2C illustrates the angling of test pad legs 230 and 235 upon extension through perforations 250 and 255, respectively. As FIG. 2C illustrates, test pad legs 230 and 235 are angled upon exiting the perforations in the carrier strip opposite the side of test pad 220. Denoted as 240 and 245, respectively, the angled test pad legs further facilitate the securing of test pad 220 to the diagnostic test strip. Furthermore, carrier strip perforations 257 and 259 form indentations in carrier strip 210 opposite the side of the carrier strip having test pad 220. Indentations 257 and 259 readily accept angled portions of the test pad legs, denoted as 247 and 249, respectively. Acceptance of test pad leg portions 247 and 249 further aids the securing of test pad 220 to the diagnostic test strip.

FIG. 2D is a schematic illustration of a perspective view of diagnostic test strip 200. In FIG. 2D, carrier strip perforations 250 and 255 form holes such that they readily accept test pad legs 230 and 235, respectively. Additionally, FIG. 2D illustrates the angling of test pad legs 230 and 235 upon extension through perforations 250 and 255, respectively. As FIG. 2D illustrates, test pad legs 230 and 235 are angled upon exiting the perforations in the carrier strip opposite the side of test pad 220. Denoted as 240 and 245, respectively, the angled test pad legs further facilitate the securing of test pad 220 to the diagnostic test strip. Furthermore, carrier strip perforations 257 and 259 form indentations in carrier strip 210 opposite the side of the carrier strip having test pad 220. Indentations 257 and 259 readily accept angled portions of the test pad legs, denoted as 247 and 249, respectively. Acceptance of test pad leg portions 247 and 249 further aids the securing of test pad 220 to the diagnostic test strip.

Referring to the drawings, FIGS. 2E-2F illustrate schematically an embodiment of a diagnostic test strip, 205, having a carrier strip, 215, and two test pads, 220 and 225. FIG. 2E is a schematic illustration of a side view of diagnostic test strip 205. In FIG. 2E, carrier strip perforations 250 and 255 form holes such that they readily accept test pad legs 230 and 235, respectively. Furthermore, carrier strip perforations 257 and 254 form holes such that they readily accept test pad legs 237 and 239, respectively. Additionally, FIG. 2E illustrates the angling of test pad legs 230 and 235 upon extension through perforations 250 and 255, respectively. As FIG. 2E illustrates, test pad legs 230 and 235 are angled upon exiting the perforations in the carrier strip opposite the side of test pad 220. Denoted as 240 and 245, respectively, the angled test pad legs further facilitate the securing of test pad 220 to the diagnostic test strip. Furthermore, carrier strip perforations 257 and 259 form indentations in carrier strip 215 opposite the side of the carrier strip having test pad 220. Indentations 257 and 259 readily accept angled portions of the test pad legs, denoted as 247 and 249, respectively. Acceptance of test pad leg portions 247 and 249 further aids the securing of test pad 220 to the diagnostic test strip. FIG. 2E also illustrates the angling of test pad legs 237 and 239 upon extension through perforations 257 and 254, respectively. As FIG. 2E illustrates, test pad legs 237 and 239 are angled upon exiting the perforations in the carrier strip opposite the side of test pad 225. Denoted as 241 and 242, respectively, the angled test pad legs further facilitate the securing of test pad 225 to the diagnostic test strip. Furthermore, carrier strip perforations 251 and 252 form indentations in carrier strip 215 opposite the side of the carrier strip having test pad 225. Indentations 251 and 252 readily accept angled portions of the test pad legs, denoted as 243 and 244, respectively. Acceptance of test pad leg portions 243 and 244 further aids the securing of test pad 225 to the diagnostic test strip.

FIG. 2F is a schematic illustration of a perspective view of diagnostic test strip 205. In FIG. 2F, carrier strip perforations 250 and 255 form holes such that they readily accept test pad legs 230 and 235, respectively. Furthermore, carrier strip perforations 257 and 254 form holes such that they readily accept test pad legs 237 and 239, respectively. Additionally, FIG. 2F illustrates the angling of test pad legs 230 and 235 upon extension through perforations 250 and 255, respectively. As FIG. 2F illustrates, test pad legs 230 and 235 are angled upon exiting the perforations in the carrier strip opposite the side of test pad 220. Denoted as 240 and 245, respectively, the angled test pad legs further facilitate the securing of test pad 220 to the diagnostic test strip. Furthermore, carrier strip perforations 257 and 259 form indentations in carrier strip 215 opposite the side of the carrier strip having test pad 220. Indentations 257 and 259 readily accept angled portions of the test pad legs, denoted as 247 and 249, respectively. Acceptance of test pad leg portions 247 and 249 further aids the securing of test pad 220 to the diagnostic test strip. FIG. 2F also illustrates the angling of test pad legs 237 and 239 upon extension through perforations 257 and 254, respectively. As FIG. 2F illustrates, test pad legs 237 and 239 are angled upon exiting the perforations in the carrier strip opposite the side of test pad 225. Denoted as 241 and 242, respectively, the angled test pad legs further facilitate the securing of test pad 225 to the diagnostic test strip. Furthermore, carrier strip perforations 251 and 252 form indentations in carrier strip 215 opposite the side of the carrier strip having test pad 225. Indentations 251 and 252 readily accept angled portions of the test pad legs, denoted as 243 and 244, respectively. Acceptance of test pad leg portions 243 and 244 further aids the securing of test pad 225 to the diagnostic test strip.

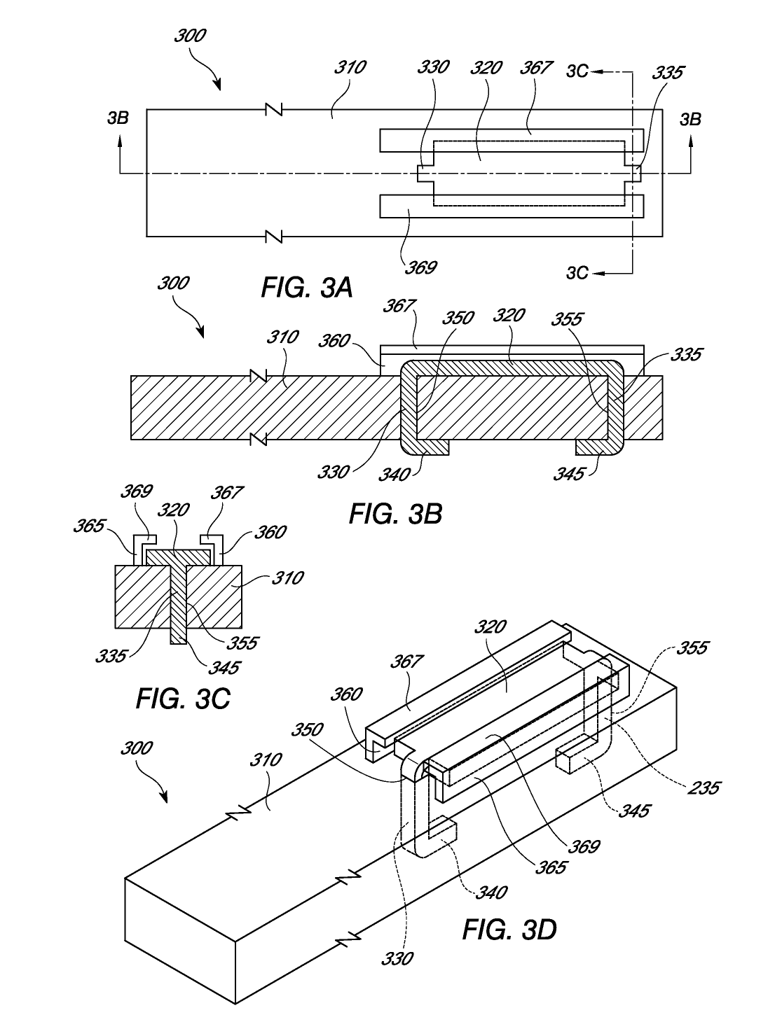

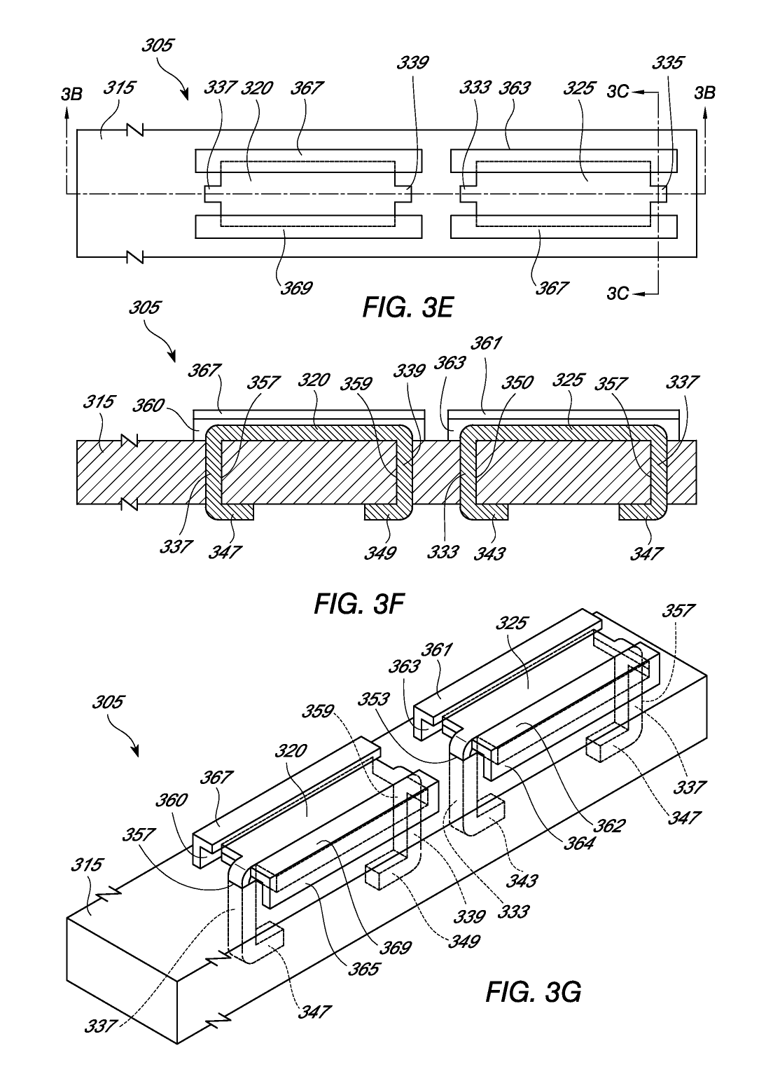

Referring to the drawings, FIGS. 3A-3D illustrate schematically an embodiment of a diagnostic test strip, 300, having a carrier strip, 310, one test pad, 320, and two protrusions located on the diagnostic test strip. FIG. 3A is a top view of an embodiment of a diagnostic test strip, 300, having one test pad, 320, secured by two test pad legs, 330 and 335. In FIG. 3A, test pad legs, 330 and 335 facilitate the securing of test pad 320 to the diagnostic test strip. Furthermore, FIG. 3A illustrates two protrusions, portions of which are designated as 360 and 365. Protrusions 360 and 365 surround two sides of test pad 320 and extend above a portion of test pad 320. Protrusions 360 and 365 protect test pad 320 from both damage and removal from the diagnostic test strip.

FIG. 3B is a schematic illustration of a side view of diagnostic test strip 300. In FIG. 3B, carrier strip perforations 350 and 355 form holes such that they readily accept test pad legs 330 and 335, respectively. Additionally, FIG. 3B illustrates the angling of test pad legs 330 and 335 upon extension through perforations 350 and 355, respectively. As FIG. 3B illustrates, test pad legs 330 and 335 are angled upon exiting the perforations in the carrier strip opposite the side of test pad 320. Denoted as 340 and 345, respectively, the angled test pad legs further facilitate the securing of test pad 320 to the diagnostic test strip. Furthermore, FIG. 3B illustrates one protrusion, portions of which are designated as 360 and 367. The second protrusion surrounding test pad 320 as represented in FIG. 3A is removed for clarity. In FIG. 3B, one can readily appreciate that protrusion 367 extends vertically from the carrier strip while protrusion 360 extends perpendicular with respect to protrusion 360 and horizontal with respect to test pad 320. Consequently, protrusions 360 and 367 surround one side of test pad 320 and protect test pad 320 from both damage and removal from the diagnostic test strip. Optionally, an embodiment may possess additional perforations and angling of the test pad legs, including, but not limited to, the additional perforations and angling illustrated in FIGS. 2A-2-F.

FIG. 3C is a schematic illustration of an end view of diagnostic test strip 300. In FIG. 3C, one can readily appreciate that test pad 320 is secured to carrier strip 210 by the acceptance of test pad leg 335 in perforation 355. Additionally, FIG. 3C illustrates the extension of test pad leg 335 through perforation 355 to afford a portion of the test pad leg 345, which is angled under the carrier strip opposite test pad 320 to facilitate the securing of the test pad to the diagnostic test strip. Furthermore, FIG. 3C illustrates two protrusions, portions of which are designated as 360 and 367, respectively, and 365 and 369, respectively. In FIG. 3C, one can readily appreciate that protrusion 367 extends vertically from the carrier strip while protrusion 360 extends perpendicular with respect to protrusion 367 and horizontal with respect to test pad 320. Similarly, protrusion 369 extends vertically from the carrier strip while protrusion 365 extends perpendicular with respect to protrusion 369 and horizontal with respect to test pad 320. Consequently, the two protrusions surround two sides of test pad 320 and protect test pad 320 from both damage and removal from the diagnostic test strip. Optionally, an embodiment may possess additional perforations and angling of the test pad legs, including, but not limited to, the additional perforations and angling illustrated in FIGS. 2A-2-F.

FIG. 3D is a schematic illustration of perspective view of diagnostic test strip 300. In FIG. 3D, carrier strip perforations 350 and 355 form holes such that they readily accept test pad legs 330 and 335, respectively. Additionally, FIG. 3D illustrates the angling of test pad legs 330 and 335 upon extension through perforations 350 and 355, respectively. As FIG. 3D illustrates, test pad legs 330 and 335 are angled upon exiting the perforations in the carrier strip opposite the side of test pad 320. Denoted as 340 and 345, respectively, the angled test pad legs further facilitate the securing of test pad 320 to the diagnostic test strip. Furthermore, FIG. 3D illustrates one protrusion, portions of which are designated as 360 and 367. In FIG. 3D, one can readily appreciate that protrusion 367 extends vertically from the carrier strip while protrusion 360 extends perpendicular with respect to protrusion 360 and horizontal with respect to test pad 320. Consequently, protrusions 360 and 367 surround one side of test pad 320 and protect test pad 320 from both damage and removal from the diagnostic test strip. Optionally, an embodiment may possess additional perforations and angling of the test pad legs, including, but not limited to, the additional perforations and angling illustrated in FIGS. 2A-2-F.

Referring to the drawings, FIGS. 3E-3G illustrate schematically an embodiment of a diagnostic test strip, 305, having a carrier strip, 315, two test pads, 320 and 325, and four protrusions located on the diagnostic test strip. FIG. 3E is a top view of diagnostic test strip 305. In FIG. 3A, test pad legs, 330 and 335 facilitate the securing of test pad 320 to the diagnostic test strip. Similarly, test pad legs 333 and 337 facilitate the securing of test pad 325 to the diagnostic test strip. Furthermore, FIG. 3A illustrates four protrusions, two protrusions surround two sides of test pad 320, and have portions designated as 360 and 365, respectively. Similarly, two protrusions surround two sides of test pad 325, and have portions designated 363 and 367, respectively. As indicated in FIG. 3E, protrusions 360 and 365 extend above a portion of test pad 320. Similarly, protrusions 363 and 367 extend above a portion of test pad 325. Collectively, these protrusions help protect test pads 320 and 325 from both damage and removal from the diagnostic test strip. Optionally, an embodiment may possess additional perforations and angling of the test pad legs, including, but not limited to, the additional perforations and angling illustrated in FIGS. 2A-2-F.

FIG. 3F is a schematic illustration of a side view of diagnostic test strip 305. In FIG. 3F, carrier strip perforations 350 and 355 form holes such that they readily accept test pad legs 330 and 335, respectively. Similarly, carrier strip perforations 353 and 357 form holes such that they readily accept test pad legs 333 and 337, respectively. Additionally, FIG. 3F illustrates the angling of test pad legs 330 and 335, and 333, and 337, respectively, upon extension through perforations 350 and 355, and 353 and 357, respectively. As FIG. 3F illustrates, test pad legs 330 and 335, and 333 and 337, respectively, are angled upon exiting the perforations in the carrier strip opposite the side of test pad 320 and 325. Denoted as 340 and 345, respectively, and 343 and 347, respectively, the angled test pad legs further facilitate the securing of test pads 320 and 325 to the diagnostic test strip. Furthermore, FIG. 3F illustrates two protrusions, with portions of one protrusion designated as 360 and 367 while portions of a second protrusion are designated 363 and 361. Two additional protrusions surrounding test pads 320 and 325 as represented in FIG. 3A are removed for clarity. In FIG. 3F, one can readily appreciate that protrusions 367 and 363 extend vertically from the carrier strip while protrusion 360 and 361 extend perpendicular with respect to protrusions 367 and 363. Consequently, two sides each of test pads 320 and 325 are surrounded, while a portion of test pads 320 and 325 are surrounded on top. Collectively, these protrusions protect test pads 320 and 325 from both damage and removal from the diagnostic test strip. Optionally, an embodiment may possess additional perforations and angling of the test pad legs, including, but not limited to, the additional perforations and angling illustrated in FIGS. 2A-2-F.

FIG. 1G is a schematic illustration of perspective view of diagnostic test strip 305. In FIG. 3F, carrier strip perforations 350 and 355 form holes such that they readily accept test pad legs 330 and 335, respectively. Similarly, carrier strip perforations 353 and 357 form holes such that they readily accept test pad legs 333 and 337, respectively. Additionally, FIG. 3F illustrates the angling of test pad legs 330 and 335, and 333, and 337, respectively, upon extension through perforations 350 and 355, and 353 and 357, respectively. As FIG. 3F illustrates, test pad legs 330 and 335, and 333 and 337, respectively, are angled upon exiting the perforations in the carrier strip opposite the side of test pad 320 and 325. Denoted as 340 and 345, respectively, and 343 and 347, respectively, the angled test pad legs further facilitate the securing of test pads 320 and 325 to the diagnostic test strip. Furthermore, FIG. 3F illustrates two protrusions, with portions of one protrusion designated as 360 and 367 while portions of a second protrusion are designated 363 and 361. Two additional protrusions surrounding test pads 320 and 325 as represented in FIG. 3A are removed for clarity. In FIG. 3F, one can readily appreciate that protrusions 367 and 363 extend vertically from the carrier strip while protrusion 360 and 361 extend perpendicular with respect to protrusions 367 and 363. Consequently, two sides each of test pads 320 and 325 are surrounded, while a portion of test pads 320 and 325 are surrounded on top. Collectively, these protrusions protect test pads 320 and 325 from both damage and removal from the diagnostic test strip. Optionally, an embodiment may possess additional perforations and angling of the test pad legs, including, but not limited to, the additional perforations and angling illustrated in FIGS. 2A-2-F.

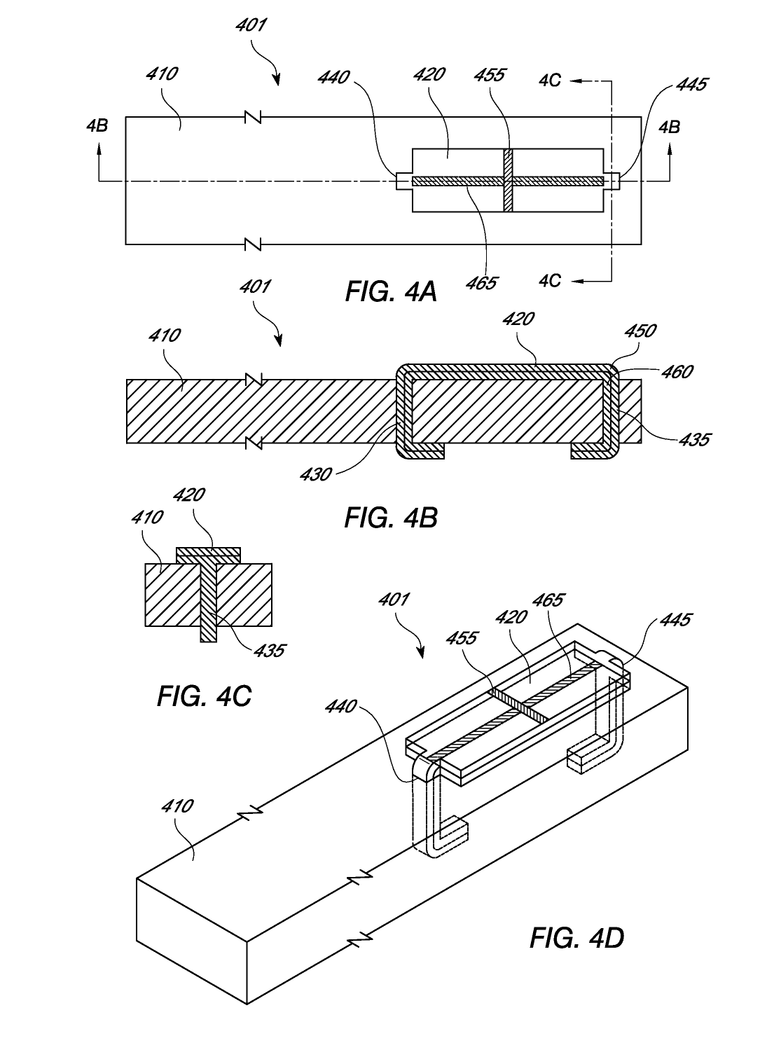

FIGS. 4A, 4B, 4C, and 4D illustrate an alternative embodiment of a diagnostic test strip, 401. FIG. 4A shows a top view of the diagnostic test strip, 401. FIG. 4B shows a cross-sectional view of the diagnostic test strip, 401, taken along the line 4B-4B in FIG. 4A. FIG. 4C shows a cross-sectional view of the diagnostic test strip, 401, taken along the line 4C-4C in FIG. 4A. FIG. 4D shows a perspective view of the diagnostic test strip, 401. In this embodiment, the diagnostic test strip, 4, includes a carrier strip, 410, and test pad, 420. In this embodiment, carrier strip, 410, includes holes, 430 and 435, and the test pad, 420, includes legs, 440 and 445. The legs, 440 and 445, extend through the holes, 430 and 435, in the carrier strip, 410. Also, in this embodiment, legs, 440 and 445, protrude through the carrier strip, 410, and bend to contact the bottom of the carrier strip, 410. Accordingly, the test pad, 420, is secured to the carrier strip, 410, by the legs, 440 and 445. Other arrangements may be practiced. Test pad 420 is illustrated as comprising at least two test pad layers, 450 and 460. Consequently, analyte detection by test pad 420 can result in the production of two or more lines resulting from signals 455 and 465. Test pad layers 450 and 460 are capable of generating signals 455 and 465 upon detection of the same analyte, different analytes, and/or different markers for the same analyte.

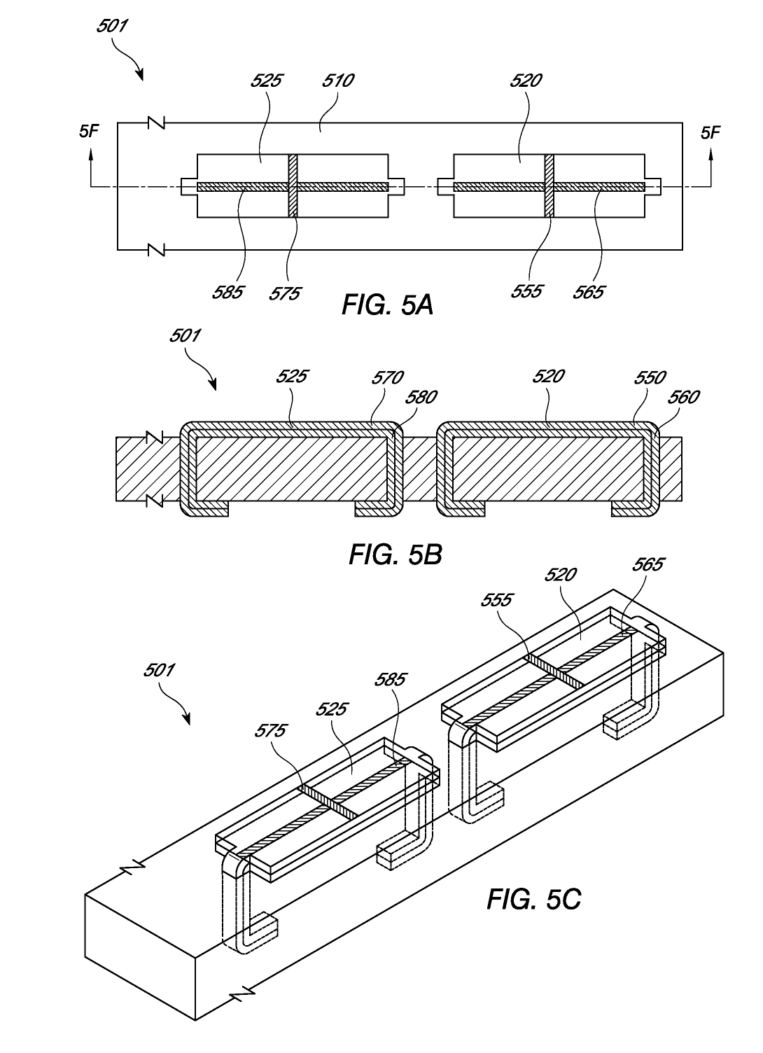

FIGS. 5A, 5B, 5C illustrate an alternative embodiment of a diagnostic test strip, 501. FIG. 5A shows a top view of the diagnostic test strip, 501. FIG. 5B shows a cross-sectional view of the diagnostic test strip, 501, taken along the line 5B-5B in FIG. 5A. FIG. 5C shows a perspective view of the diagnostic test strip, 501. In this embodiment, the diagnostic test strip, 501, includes a carrier strip, 510, and test pads, 520 and 525. In this embodiment, test pads, 520 and 525, may be held in place by mechanisms similar to those described above with reference to FIGS. 4A, 4B, 4C, and 4D. Other arrangements may be practiced. Test pad 520 is illustrated as comprising at least two test pad layers, 550 and 560. Consequently, analyte detection by test pad 520 can result in the production of two or more lines resulting from signals 555 and 565. Test pad layers 550 and 560 are capable of generating signals 555 and 565 upon detection of the same analyte, different analytes, and/or different markers for the same analyte. Test pad 525 is illustrated as comprising at least two test pad layers, 570 and 580. Consequently, analyte detection by test pad 525 can result in the production of two or more lines resulting from signals 575 and 585. Test pad layers 570 and 580 are capable of generating signals 575 and 585 upon detection of the same analyte, different analytes, and/or different markers for the same analyte.

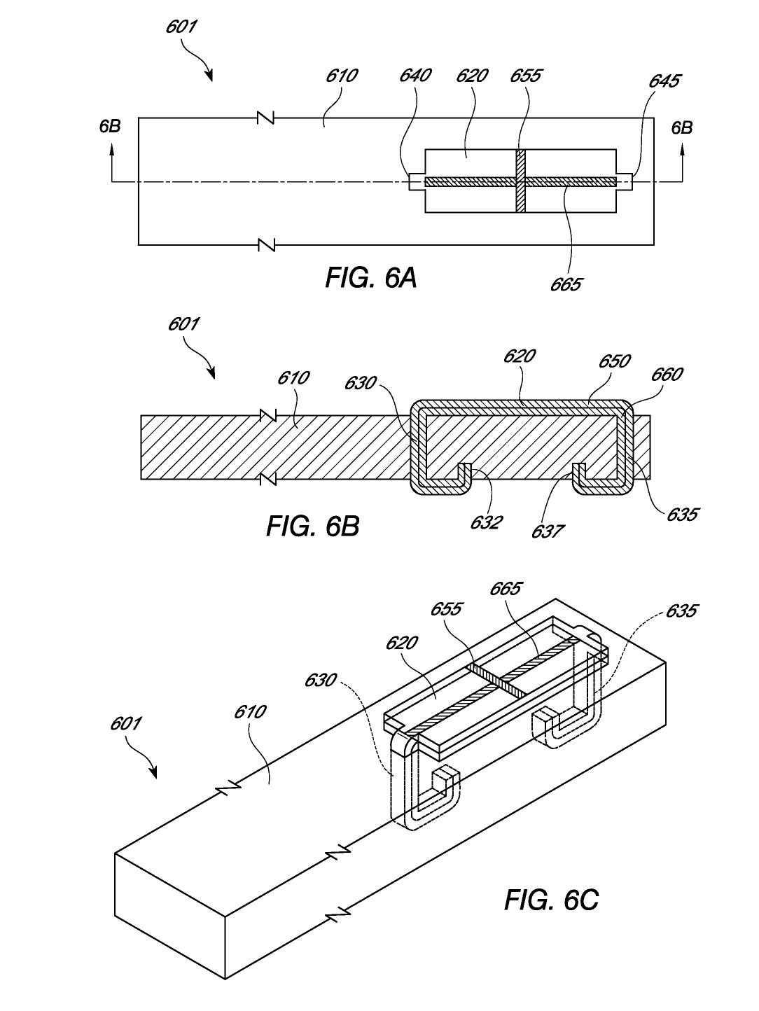

FIGS. 6A, 6B, 6C illustrate an alternative embodiment of a diagnostic test strip, 601. FIG. 6A shows a top view of the diagnostic test strip, 601, FIG. 6B shows a cross-sectional view of the diagnostic test strip, 601, taken along the line 6B-6B in FIG. 6A. FIG. 6C shows a perspective view of the diagnostic test strip, 601. In this embodiment, the diagnostic test strip, 601, includes a carrier strip, 610, and test pads, 620 and 625. In this embodiment, carrier strip, 610, includes holes, 630, 632, 635, and 637, and the test pad, 620, includes legs, 640 and 645. The legs, 640 and 645, extend through the holes, 630 and 635, in the carrier strip, 610. Also, in this embodiment, legs, 640 and 645, protrude through the carrier strip, 610, bend to contact the bottom of the carrier strip, 610, and extend into the holes, 632 and 637. Accordingly, the test pad, 620, is secured to the carrier strip, 610, by the legs, 640 and 645. Other arrangements may be practiced. Test pad 620 is illustrated as comprising at least two test pad layers, 650 and 660. Consequently, analyte detection by test pad 620 can result in the production of two or more lines resulting from signals 655 and 665. Test pad layers 650 and 660 are capable of generating signals 655 and 665 upon detection of the same analyte, different analytes, and/or different markers for the same analyte.

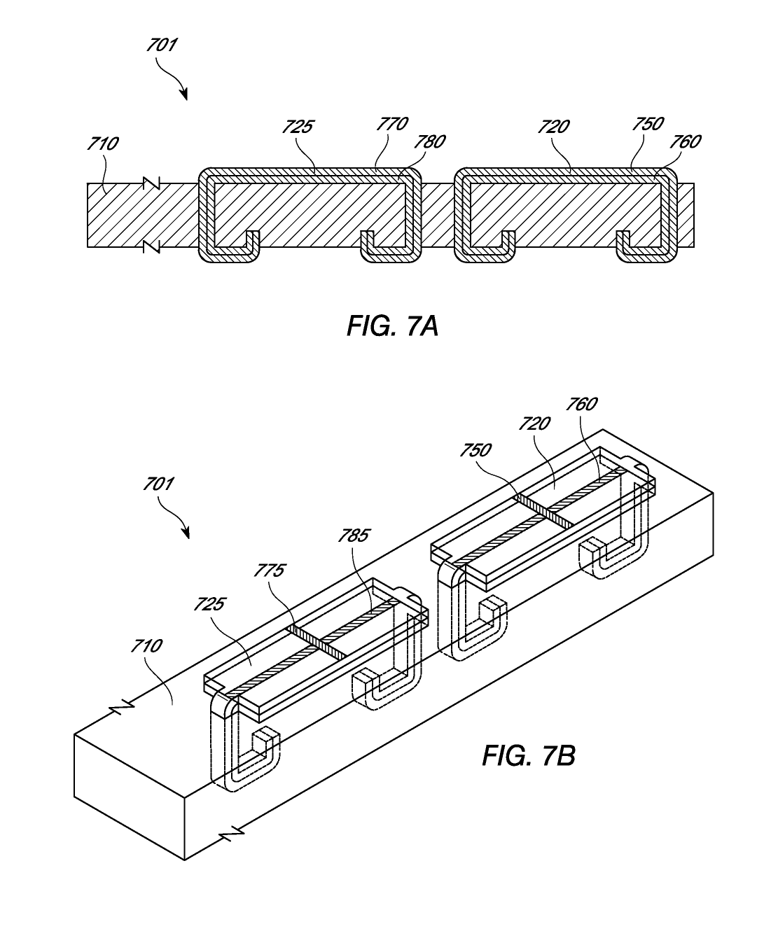

FIGS. 7A and 7B illustrate an alternative embodiment of a diagnostic test strip, 701. FIG. 7A shows a cross-sectional view of the diagnostic test strip, 701, and FIG. 7B shows a perspective view of the diagnostic test strip, 701. In this embodiment, the diagnostic test strip, 701, includes a carrier strip, 710, and test pads, 720 and 725. In this embodiment, test pads, 720 and 725, may be held in place by mechanisms similar to those described above with reference to FIGS. 6A, 6B, and 6C. Other arrangements may be practiced. Test pad 720 is illustrated as comprising at least two test pad layers, 750 and 760. Consequently, analyte detection by test pad 720 can result in the production of two or more lines resulting from signals 755 and 765. Test pad layers 750 and 760 are capable of generating signals 755 and 765 upon detection of the same analyte, different analytes, and/or different markers for the same analyte. Test pad 725 is illustrated as comprising at least two test pad layers, 770 and 780. Consequently, analyte detection by test pad 725 can result in the production of two or more lines resulting from signals 775 and 785. Test pad layers 770 and 780 are capable of generating signals 775 and 785 upon detection of the same analyte, different analytes, and/or different markers for the same analyte.

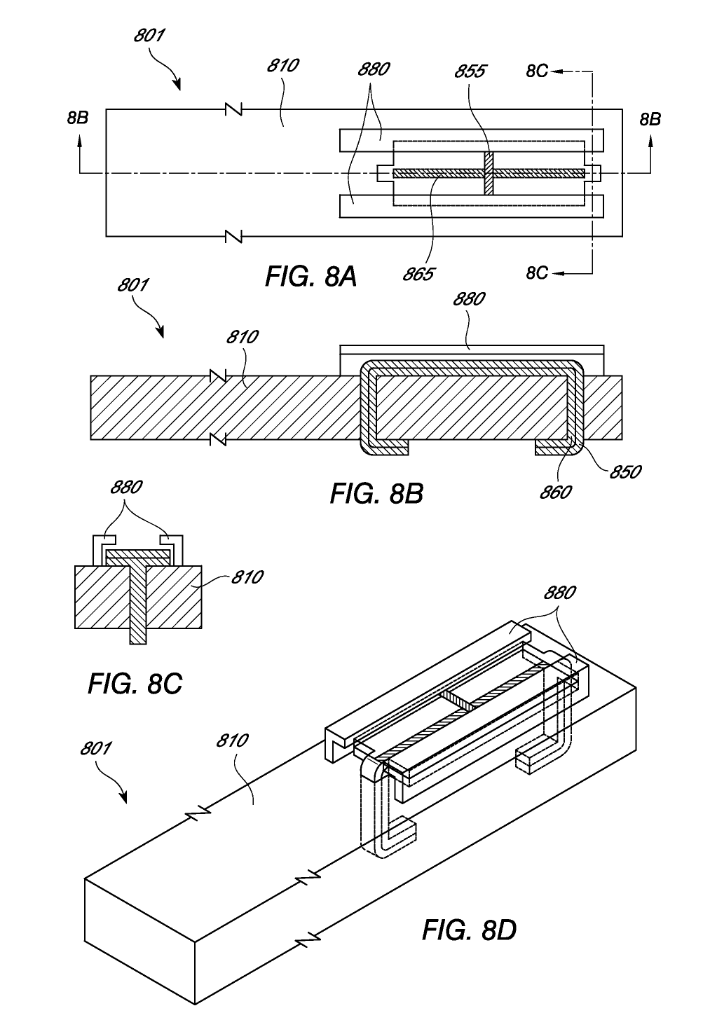

FIGS. 8A, 8B, 8C, and 8D illustrate an alternative embodiment of a diagnostic test strip, 801. FIG. 8A shows a top view of the diagnostic test strip, 801. FIG. 8B shows a cross-sectional view of the diagnostic test strip, 801, taken along the line 8B-8B in FIG. 8A. FIG. 8C shows a cross-sectional view of the diagnostic test strip, 801, taken along the line 8C-8C in FIG. 8A. FIG. 8D shows a perspective view of the diagnostic test strip, 801. In this embodiment, test pad, 820, may be held in place by mechanisms similar to those described above with reference to FIGS. 6A, 6B, and 6C. This embodiment includes a carrier strip, 810, a test pad, 820, and shields, 880, which provide mechanical protection to the test pad, 820. The shields, 880, are attached to the carrier strip, 810, and extend from carrier strip, 810, farther than the test pad, 820. Other arrangements may be practiced. Test pad 820 is illustrated as comprising at least two test pad layers, 850 and 860. Consequently, analyte detection by test pad 820 can result in the production of two or more lines resulting from signals 855 and 865. Test pad layers 850 and 860 are capable of generating signals 855 and 865 upon detection of the same analyte, different analytes, and/or different markers for the same analyte.

In alternative embodiments, the legs of the test pads extend only partially into the carrier strip. In some embodiments, the test pad is substantially square or substantially circular. In addition, the test pad may have one leg or more than two legs, for example, three or four legs. In some embodiments, at least one test pad is substantially at one end of the carrier strip. In some embodiments, a plurality of test pads are placed sequentially over the length of the carrier strip with a space separating each test pad on the carrier strip.

### Carrier Strip

The carrier strip provides structural support for the one or more test pads and the one or more optional protrusions. As a structural support, many materials suitable for use in preparing the carrier strip are known in the art. Such materials include but are not limited to plastics including polyethylene terephthalate, high-density polyethylene, polypropylene, cellulose, Bakelite, polystyrene, high impact polystyrene, acrylonitrile butadiene styrene, polyester, polyurethanes, polycarbonates, polycarbonate/acrylonitrile butadiene styrene, polymethyl methacrylate, polytetrafluoroethylene, polyetherimide, phenol formaldehydes, urea-formaldehyde, melamine formaldehyde, polylactic acid, plastarch material, polyvinylchloride, nylon, and other polyamides, metals, alloys, ceramics, glass, wood, cardboard, paper, natural rubber, synthetic rubber, and other suitable polymers. Optionally, the carrier strip may be porous or non-porous. Optionally, the carrier strip may facilitate the transmission of information from the one or more test pads to a memory device. Transmitted information may include, but is not limited to, the presence, absence, and/or concentration of one or more analytes of interest. The carrier strip may facilitate the transmission of information from the one or more test pads to the one or more memory devices by any of several methods known in the art. Such methods include, but are not limited to, the transmission of electrical signals which result from changes in the coulometry, amperometry, or potentiometry of the materials comprising the carrier strip. See U.S. Pat. No. 6,743,635 (Neel et al., issued on Jun. 1, 2001) and U.S. Pat. No. 6,946,299 (Neel at al., issued on Sep. 20, 2005), which are herein incorporated by reference. Alternatively, the carrier strip may facilitate the transmission of optical signals which result from differences in the reflection, transmission, scattering, absorption, fluorescence, or electrochemiluminescense of the materials comprising the carrier strip and/or the test pads. See U.S. Pat. No. 6,040,195 (Carroll et al., issued on Mar. 21, 2000) and U.S. Pat. No. 6,284,550 (Carroll et al., issued on Sep. 4, 2001) which are herein incorporated by reference.

The carrier strip's size and shape is only limited by the desired application of the embodiment. For example, if the desired application is testing a human patient, the embodiment, and consequently the carrier strip, may be smaller or larger depending upon the size of the human patient. Likewise, if the desired application involves testing an animal patient, the embodiment, and consequently the carrier strip, may be smaller or larger depending upon the size of the animal patient. In some embodiments, the carrier strip is about 1, about 1.25, about 1.5, about 1.75, about 2, about 2.25, about 2.5, about 2.75, about 3, about 3.25, about 3.5, about 3.75, about 4, about 1-2, about 1-3, about 1-4, about 2-3, about 2-4, or about 3-4 inches in length. The carrier strip's shape may optionally be varied depending upon the desired application of the embodiment. Some applications may require substantially narrow, fat, rectangular, circular, oval, square, triangular, or other shapes, including combinations of the indicated shapes. It is envisioned that the shape of embodiments can be tailored to the shape of the environment in which the embodiments will be applied. Moreover, the carrier strip may contain boundary projections that substantially surround one, two, three, and/or four sides of one or more test pads to collect and/or direct sample application to the one or more test pads. Furthermore, it is envisioned that a handle may be optionally attached to a carrier strip or in contact with a carrier strip, either directly or indirectly.

In some embodiments, the carrier strip may have one or more perforations. In some embodiments, the one or more perforations form one or more holes through the carrier strip. In other embodiments, the one or more perforations form one or more indentations in the carrier strips. In some embodiments, combinations the perforations include both holes through the carrier strip and indentations in the carrier strip. The perforations in the carrier strip may be of any size and shape suitable for accepting legs extending from test pads. As non-limiting examples, the perforations may be substantially cylindrical, rectangular, square, oval, or any other desired shape.

In other embodiments, the carrier strip may comprise one or more layers. In such embodiments, one or more layers of the carrier strip may be perforated such that the one or more layers may accept test pad legs. In such embodiments, layers of carrier strip may be contacted such that the one or more test pads are secured by sandwiching portions of the one or more test pad lags between two or more layers of carrier strip.

Perforations in the carrier strip may be obtained by any method known in the art. As non-limiting examples, carrier strips may have their perforations introduced using pins, needles, die and punch, laser, molding, or extrusion. Pins and needles can be used cold or heated, and cold perforation tools include, but are not limited to, needle punches.

### Protrusions

Optionally, protrusions surround portions of the one or more test pads. In some embodiments, protrusions extend perpendicular to the carrier strip and surround one or more, two or more, three or more, or four or more sides of the one or more test pads. In some embodiments, the protrusions extend perpendicular to the carrier strip and surround all sides of the one or more test pads. In other embodiments, the protrusions surround portions of the face of the test pads opposite the carrier strip by extending perpendicular to protrusions that are perpendicular to the carrier strip. In some embodiments, about 1-20%, about 1-40%, about 1-60%, about 1-80%, about 10-80%, about 20-60%, about 30-50% of the surface area of the face of the test pads is covered by protrusions.

Many materials suitable for use in preparing the protrusions are known in the art. Such materials include but are not limited to plastics including polyethylene terephthalate, high-density polyethylene, polypropylene, cellulose, Bakelite, polystyrene, high impact polystyrene, acrylonitrile butadiene styrene, polyester, polyurethanes, polycarbonates, polycarbonate/acrylonitrile butadiene styrene, polymethyl methacrylate, polytetrafluoroethylene, polyetherimide, phenol formaldehydes, urea-formaldehyde, melamine formaldehyde, polylactic acid, plastarch material, polyvinylchloride, nylon, and other polyamides, metals, alloys, ceramics, glass, wood, cardboard, paper, natural rubber, synthetic rubber, and other suitable polymers. Optionally, the protrusions may be porous or non-porous.

### Test Reagents and Signaling Reagents

Test reagents and signaling reagents suitable for inclusion in embodiments are well known in the art. Such reagents include, but are not limited to, polyclonal antisera and monoclonal antibodies that have specific binding properties and high affinity for virtually any antigenic substance. Literature affords many means of preparing such reagents. See, e.g., Laboratory Techniques in Biochemistry and Molecular Biology, Tijssen, Vol. 15, Practice and Theory of Enzyme Immunoassays, chapter 13, The immobilization of Immunoreactants on Solid Phases, pp. 297-328, and the references cited therein which are herein incorporated by reference. Additional assay protocols, reagents, and analytes useful in the practice of the invention are known per se. See, e.g., U.S. Pat. No. 4,313,734 (Leuvering, issued on Feb. 2, 1982), columns 4-18, and U.S. Pat. No. 4,366,241 (Tom et al., issued on Dec. 28, 1982), columns 5-40 which are herein incorporated by reference.

Metal sols, including but not limited to gold sol, and other types of colored particles, including but not limited to, organic dye sols and colored latex particles, that are useful as marker substances in immunoassay procedures are also known per se and suitable for use as test reagents and/or signaling reagents. See, for example, U.S. Pat. No. 4,313,734 (Leuvering, issued on Feb. 2, 1982), the disclosure of which is incorporated herein by reference. For details and engineering principles involved in the synthesis of colored particle conjugates see Horisberger, Evaluation of Colloidal Gold as a Cytochromic Marker for Transmission and Scanning Electron Microscopy, Biol. Cellulaire, 36, 253-258 (1979); Leuvering et al, Sol Particle Immunoassay, J. Immunoassay 1 (1), 77-91 (1980), and Frens, Controlled Nucleation for the Regulation of the Particle Size in Monodisperse Gold Suspensions, Nature, Physical Science, 241, pp. 20-22 (1973) which are herein incorporated by reference.

Test reagents for inclusion in the embodiments may signal directly, such as with an electrical or optical signal (visible either to the naked eye, or with an optical filter or upon applied stimulation to promote fluorescence or phosphorescence). Test reagents may also signal indirectly such as with enzymes, e.g. alkaline phosphatase and/or horseradish peroxidase, in combination with signaling reagents in the form of enzymatic substrates that will generate a signal upon interaction with the enzyme. In some embodiments, the signaling reagent and/or test reagent is incorporated into the test pad. In other embodiments, the signaling reagent and/or test reagent is added to the test sample before application to the test pad. In additional embodiments, the signaling reagent and/or test reagent is added to the test pad after introduction of the test sample.

Alcohol sensitive test reagents and methods are well known in the art. See, e.g. U.S. Pat. No. 5,563,073 (Titmas, issued on Oct. 8, 1996) and Jai Moo Shin et al., Simple Diagnostic Tests to Detect Toxic Alcohol Intoxications, NIH (October 2008), which are hereby incorporated by reference in their entirety. In some embodiments, the test reagent and/or signaling reagent from Alco Screen™ pads, manufactured by Chematics, Inc. located in North Webster, Ind., is incorporated. Optionally, the test reagent and/or signaling reagent from Alco Screen™ pads is incorporated in the one or more test pads, but it may also be applied to the test pad after sample application or it may be applied to the sample before application to the test pad. In some embodiments the test reagent and/or signaling reagent from the alcohol dehydrogenase method (ADH method) is incorporated in the one or more test pads, but it may also be applied to the test pad after sample application or it may be applied to the sample before application to the test pad. In some embodiments the test reagent and/or signaling reagent from the alcohol oxidase method (ALOx method) is incorporated in the one or more test pads, but it may also be applied to the test pad after sample application or it may be applied to the sample before application to the test pad. In some embodiments the test reagent and/or signaling reagent from the sodium periodate method is incorporated in the one or more test pads, but it may also be applied to the test pad after sample application or it may be applied to the sample before application to the test pad. In some embodiments the test reagent and/or signaling reagent from the potassium permanganate method (PA method) is incorporated in the one or more test pads, but it may also be applied to the test pad after sample application or it may be applied to the sample before application to the test pad.

Test reagents and/or signaling reagents may also detect the storage and handling of embodiments. In some embodiments, test reagents and/or signaling reagents may be sensitive to temperature and if the temperature of the embodiment's environment has exceeded or fallen below a predetermined temperature, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

In some embodiments, test reagents and/or signaling reagents may be sensitive to moisture, and if the humidity of the embodiment's environment has exceeded or fallen below a predetermined level, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

Test reagents and/or signaling reagents may also detect whether a sufficient amount of sample has been applied to an embodiment for analysis. For example, when the sample is saliva, a test reagent and/or signaling reagent specific for a salivary enzyme, such as amylase, may detect the salivary enzyme's presence if a sufficient volume of sample has been applied. The detection of a sufficient sample may optionally be signaled to the user in the form of a color or symbol. Using such embodiments, the user would then know if a sufficient quantity of sample was applied to the one or more test pads to afford an accurate analysis.

Embodiments that detect storage and/or sufficient application of sample volume are particularly capable of reducing the occurrence of false negatives. For example, poor storage conditions may inactivate a test reagent in a test pad. Upon application of sample to such a test pad, no signal may result and a user could believe that an analyte is not present—a false negative. Alternatively, test pads having a pre-printed negative signal may suffer a similar occurrence of a false negative if the test reagent is inactivated because an analytes presence in a sample would not convert the pre-printed negative signal into a positive signal. Likewise, an insufficient volume of sample may generate no signal or a negative signal and cause a user to believe that an analyte is not present.

Any enzyme, antibody, dye buffer, chemical, sol, or combinations thereof may be incorporated so long as the enzyme, antibody, dye buffer, chemical, metal sol, or combinations thereof are capable of detecting the presence of one or more analytes in a sample. See, e.g., U.S. Pat. No. 6,383,736 (Titmas, issued on May 7, 2002), U.S. Pat. No. 7,858,756 (Owens et al., issued on Dec. 28, 2010), and U.S. Pat. No. 7,790,400 (Jehanli et al., issued on Sep. 7, 2010) which are hereby incorporated by reference in their entirety.

### Test Pads

The one or more test pads may be prepared from any bibulous, porous, fibrous, or sorbent material capable of rapidly absorbing a sample. Porous plastics material, such as polypropylene, polyethylene, polyvinylidene fluoride, ethylene vinylacetate, acrylonitrile and polytetrafluoroethylene can be used. Optionally, the one or more test pads can be pre-treated with a surface-active agent to reduce any inherent hydrophobicity in the one or more test pads and enhance their ability to absorb a sample. Moreover any one of the one or more test pads may be treated with an oxygen-impermeable water soluble substance. Suitable examples of an oxygen-impermeable water soluble substance include, but are not limited to, polyvinyl alcohol, partly saponified polyvinyl acetate which can also contain vinylether and vinylacetal units, polyvinyl pyrrolidone and copolymers thereof with vinyl acetate and vinyl ethers, hydroxy alkyl cellulose, gelatin, polyacrylic acid, gum arabic, polyacryl amide, dextrin, cyclodextrin, copolymers of alkylvinyl ethers and maleic acid anhydride, ring opened polymers of maleic acid anhydride, water-soluble high molecular polymers of ethylene oxide having molecular weights of above 5,000, and/or polyvinyl alcohol in combination with poly(l-vinylimidazole) or a copolymer of 1-vinyl-imidazole. The one or more test pads can also be made from paper or other cellulosic materials, including but not limited to nitrocellulose. Materials that are now used in the nibs of fiber-tipped pens are also suitable for incorporation in the one or more test pads.

Optionally, the one or more test pads may be prepared from non-porous materials. In such circumstances, the test reagents and/or signaling reagents may be coated on the outer surface of the one or more test pads such that contact with a sample containing an analyte will result in the generation of a signal.

Using known methods, test pads may be shaped or extruded in a variety of lengths and cross-sections. Embodiments may possess one or more test pads of various sizes and shapes, and the size and shape of the one or more test pads are only limited by their number, size, and desired application of the embodiment in which they are incorporated within. In some embodiments, the one or more test pads are substantially similar in size and/or shape. In other embodiments, the one or more test pads may differ substantially in size and/or shape. It is readily envisioned that embodiments may possess about one or more test pads, about two or more test pads, about three or more test pads, about four or more test pads, about five or more test pads, about six or more test pads, about seven or more test pads, about eight or more test pads, about nine or more test pads, about ten or more test pads, about 1-4 test pads, about 1-10 test pads about 1-100 test pads, about 2-100 test pads, about 3-100 test pads, about 4-100 test pads, about 5-100 test pads, about 5-75 test pads, about 10-50 test pads, about 15-25 test pads, and individual numbers of test pads therein. The one or more test pads may be made of the same material, or optionally they may be made of different materials or even combinations of different materials. Moreover, the one or more test pads may be recessed into the carrier strip.

In some embodiments, the test pads have one or more protrusions, or legs, shaped such that the perforations in the carrier strips will readily accept the test pad legs. In some embodiments, the test pads have about one or more, about two or more, about three or more, about four or more, about five or more, about six or more, about seven or more, about eight or more, about nine or more, about ten or more, about 1-4, about 1-10, about 1-100, about 2-100, about 3-100, about 4-100, about 5-100, about 5-75, about 10-50, about 15-25, and individual numbers therein, of test pad legs. Test pad legs may constitute any shape that will be accepted by the one or more perforations in the carrier strip. Such shapes include, but are not limited to substantially cylindrical, rectangular, square, oval, or any other desired shape that matches or is accepted by the one or more perforations in the carrier strip. In some embodiments, the test pad legs will protrude through the hole-type perforations in the carrier strip. In such embodiments, the test pad legs may further secure the test pad to the carrier strip by being angled. In some embodiments, an adhesive substance may be applied to the legs to aid the adherance of the test pad to the carrier strip. In some embodiments, the perforation in the carrier strip will be indentations that do not form a hole in the carrier strip. In such embodiments, an adhesive substance may be applied to the legs to aid the adherence of the test pad to the carrier strip. In some embodiments, the adhesive substance may be applied to the test pad legs. In other embodiments, the adhesive substance may be applied to the carrier strip. By applying the adhesive substance to the test pad legs or to the perforations in the carrier strip, contact of the adhesive substance with the test pad is reduced and contamination of the test pad is therefore less likely.

In some embodiments, test pads may be prepared from a single layer of material. In other embodiments, test pads may be prepared from multiple layers of material. It is readily envisioned that embodiments may possess about one or more layers, about two or more layers, about three or more layers, about four or more layers, about five or more layers, about six or more layers, about seven or more layers, about eight or more layers, about nine or more layers, about ten or more layers, about 1-4 layers, about 1-5 layers, about 1-6 layers, about 1-7 layers, about 1-8 layers, about 1-9 layers, about 1-10 layers, about 1-100 layers, about 2-100 layers, about 3-100 layers, about 4-100 layers, about 5-100 layers, about 5-75 layers, about 10-50 layers, about 15-25 layers, and individual numbers of layers therein.

The test pad layers may be of the same or different materials. Test reagents and/or signaling reagents may also be impregnated in a single layer of material or in multiple layers of material. The impregnation may take any suitable form, including, but not limited to, a substantially uniform impregnation or impregnation with dots or stripes. Test reagents and/or signaling reagents can be impregnated in various concentrations in one or more of the multiple layers to tailor the sensitivity of the test pads to certain analytes. Such sensitivity could afford information about the concentration of an analyte in the sample. Furthermore, the impregnation may optionally be conducted in a manner that will generate a signal observable by the user upon application of a sufficient quantity of sample, detection of an analyte, or proper/improper storage of the embodiment.

When one or more test pads are comprised of multiple layers of material, one or more layers of material may be impregnated (e.g. pre-printed) with an inert chemical such that a line or “minus sign” is displayed to the user. In some embodiments, the line or “minus sign” could be in the form of a material covering the one or more test pads to give a visual impression of a line or “minus sign” on the one or more test pads. One or more additional layers of the material comprising the one or more test pads could then be impregnated with a test reagent and/or a signaling reagent that upon detecting a sufficient quantity of sample, appropriate storage temperature, and/or the presence of an analyte, the impregnated test reagent and/or signaling reagent will create a perpendicular line such that a “plus sign” will be signaled to the user. In other embodiments, the line or “minus sign” displayed in the one or more test pads could be obscured by color or opaqueness when a test reagent and/or a signaling reagent detects a sufficient quantity of sample, appropriate or inappropriate storage temperature, and/or the presence of an analyte.

The test pad layers may comprise optically transparent membranes. Detection on an analyte may then generate a signal that is opaque, partially transparent, or completely transparent. Moreover, test pad layers may be only partially optically transparent prior to application of a sample. Alternatively, the application of a sample to one or more test pad layers may result in the layers becoming optically transparent, thereby allowing a user to see generated and/or pre-printed signals on test pad layers below the optically transparent layers. Moreover, the individual layers in a test pad may be positioned such that the detection of an analyte in a lower layer of material is obscured by the detection of an analyte in a layer of material positioned above the lower layer.

It is also envisioned that embodiments may have arrangements of test pads and/or arrangements of layers within multiple layered test pads such that the detection of an analyte in the test pads or the layers of a test pad generate a signal, such as a “plus sign” or “minus sign” to the user. Such embodiments may comprise at least two layers of material, each capable of generating a line upon detecting an analyte or a certain concentration of an analyte. Optionally, the lines may intersect to generate a “plus” sign or other signal upon the detection of an analyte in the at least two layers of material. Alternatively, embodiments may comprise at least four layers of material, each capable of generating a line upon detecting an analyte or a certain concentration of an analyte in the at least four layers of material. Optionally, the lines may intersect at one or more points such that a “plus” sign or other symbol is formed. While the aforementioned embodiments have been discussed with reference to “minus” and “plus” signs, it is envisioned that any symbol, including color changes, could be used to convey similar information to a user. Such symbols include, but are not limited to, circles, ovals, squares, triangles, trapezoids, rhombi, plus signs, minus signs, “X” shaped signs, checkmarks, and/or dotted, dashed, or differentially colored version of said symbols. The meaning of any desired symbol or color change could be included in the packaging of an embodiment or imprinted on an embodiment.

The test reagents applied to each layer of material may optionally be the same or different. When different test reagents are applied to different layers of material comprising the one or more test pads, the test pad may be tailored to generate a signal indicating the diagnosis of one or more illnesses, diseases, or injuries. One method for achieving such a diagnosis would be to have the individual layers comprising the test pad generate a signal in response to one or more symptoms of one or more illnesses, diseases, or injuries. For example, if the diagnosis of one or more illnesses, diseases, or injuries required the determination of multiple analytes, then the detection of each analyte could produce a portion of a symbol that is visible to the user. Upon formation of a complete symbol, the embodiment would confirm the presence of a certain illness, disease, or injury. Optionally, information relating to each specific analyte could be transferred to the one or more memory devices.

One can readily appreciate the application of such embodiments of multiple layer test pads when knowledge of a certain concentration is needed. As a non-limiting application, the detection of a person's blood alcohol level may be achieved using such an embodiment. For a test pad comprising at least four test pad layers, if a first test pad layer was sensitive to a blood alcohol level of at least 0.02%, a second test pad layer was sensitive to a blood alcohol level of at least 0.04%, a third test pad layer was sensitive to a blood alcohol level of at least 0.06%, and a fourth test pad layer was sensitive to a blood alcohol level of at least 0.08%, then the application of a sample having a blood alcohol level at least at the sensitive percentages would generate a signal. Assuming that operating a motor vehicle with a blood alcohol level equal to or greater than 0.08% is illegal, then the application of a sample that generates a “plus” sign would indicate that the sample provider should not legally operate a motor vehicle. One will readily appreciate that this described example is capable of extension to any number of test pads having any number of layers, such that the detection of an analyte in each layer generates a signal indicative of concentration.

As another non-limiting example, test reagents and/or signaling reagents that are sensitive to markers specific for hepatitis and/or liver damage may be applied to test pads and/or layers within test pads. Consequently, the detection of markers specific for hepatitis and/or liver damage in each test pad and/or layers within test pads would generate a signal. An individual test pad may optionally be sensitive to a single marker for hepatitis and/or liver damage. Alternatively, a single test pad may be sensitive to multiple markers for hepatitis and/or liver damage. In such an embodiment, the detection of one or more markers for hepatitis and/or liver damage may produce a certain signal, e.g. color, indicative of the number of markers detected and/or indicative of the exact marker detected. Alternatively, an embodiment may produce a signal in the form of a shape that indicates the presence of one or more markers indicative of hepatitis and/or liver damage. For example, an embodiment may have a test pad with four or more test pad layers, while each layer may be sensitive to one or more markers specific to an analyte such as viral hepatitis. The respective detection of a marker in each of the test pad would generate a signal such that the detection of a marker in each of the test pad layers would confirm the diagnosis of a viral hepatitis. Although such an embodiment has been described with specific references to a viral hepatitis, it is envisioned that such an embodiment may readily be tailored to detect any number of analytes and/or markers that are specific to any analyte described below.

Embodiments may optionally possess one or more test pads and test reagents that detect analytes important to a certain age population (e.g. infants, children, young adults, adults, or elderly individuals). It is also envisioned that embodiments could possess one or more test pads and test reagents that detect analytes important to certain categories of individuals (e.g., law enforcement agents, government employers, military members, chronic drug users, physicians, veterinarians, dentists, parents, private sector employers, aid workers, inmates, hospital patients, nursing home patients, outdoorsmen, immuno-compromised individuals, or students). Embodiments may also be directed to analytes important to geographic regions (e.g. third-world countries, developed countries, or specific climate regions). Such embodiments of the invention simplify the number of different embodiments that a user must purchase or travel with because users can select embodiments that will detect the analytes the users are most interested in, or are most pertinent to a user's current or impending circumstances.

In one embodiment, a single test pad contains or has applied to it a single test reagent and/or signaling reagent suitable for detecting a single analyte. In another embodiment, two or more test pads contain or have applied to one or more of them a single test reagent and/or signaling reagent suitable for detecting a single analyte. Optionally, the single test reagent and/or signaling reagent on or applied to the two or more test pads may be the same or different. Furthermore, when different test reagents and/or signaling reagents are used, the test reagents may be sensitive to the same marker on an analyte or the test reagents may be sensitive to different markers on an analyte. The analyte may optionally be the same or different. When different analytes and different test reagents and/or signaling reagents are used, the analytes and test reagent and/or signaling reagents may be tailored to detect different symptoms of the same illness, disease, or injury. In some embodiments, a diagnosis can be made based upon the detection of all the symptoms specific to an illness, disease, or injury. In other embodiments, a diagnosis can be made based upon the absence of one or more analytes specific to an illness, disease, or injury. Using these described test pads, it is readily apparent that the reduction of false negatives and false positives can be achieved by including redundancy in the embodiments.

In one embodiment, a single test pad may contain or have applied to it two or more reagents suitable for detecting and/or signaling a single analyte. These two or more test reagents and/or signaling reagents may be sensitive to the same marker of an analyte. Optionally, these two or more reagents may be sensitive to different markers on the same analyte. In some embodiments, the two or more test reagents and/or signaling reagents may be applied to the same region of the test pad. In other embodiments, the two or more test reagents and/or signaling reagents may be applied to different regions of the same test pad. The number of test reagents and/or signaling reagents suitable for incorporation or application to a single test pad is limited only by the application of the diagnostic test strip. It is readily envisioned that embodiments may possess about one or more, about two or more, about three or more, about four or more, about five or more, about six or more, about seven or more, about eight or more, about nine or more, about ten or more, about 1-4, about 1-10, about 1-100, about 2-100, about 3-100, about 4-100, about 5-100, about 5-75, about 10-50, about 15-25, and individual numbers therein, of test reagents and/or signaling reagents incorporated or applied to one or more test pads. Using these described test pads, it is readily apparent that the reduction of false negatives and false positives can be achieved by including redundancy in the embodiments.

The one or more test pads suitable for use in an embodiment will readily detect analytes present in liquid samples, such as saliva. It is also envisioned that a test pad may be capable of detecting an analyte present in solid and/or semi-solid samples. When solid and/or semi-solid samples are analyzed, it is understood that a liquid may optionally be applied to the test pad to facilitate analysis.

When liquids and/or liquid samples are applied to test pads, lateral flow through material may result from surface tension, cohesion, adhesion, wicking, and/or capillary action. In general, embodiments that utilize lateral flow will require substantial amounts of a liquid sample for sufficient contacting of the sample with a devices test area. In some embodiments, lateral flow is confined to the test pad region. In other embodiments, lateral flow is confined to individual test pads. In further embodiments, lateral flow is confined to individual layers of a multi-layer test pad. Moreover, some embodiments overcome the use of lateral flow by having a test pad designed to absorb the fluid sample without requiring surface tension, cohesion, adhesion, wicking, and/or capillary action to contact the fluid sample with the test area. Such embodiments are particularly suited for use when the volume of a fluid sample is small and/or limited. This includes, but is not limited to, instances when the fluid sample is oral fluid such as saliva.

### Analytes

An assay based on the principles described herein can be used to determine a wide variety of analytes by choice of appropriate test reagents and/or signaling reagents. The embodiments described herein can be used to test for the existence of analytes including, but not limited to, drugs, especially drugs of abuse; heavy metals; pesticides; pollutants; proteins; polynucleotides such as DNA, RNA, rRNA, tRNA, mRNA, and siRNA; hormones; vitamins; microorganisms such as bacteria, fungi, algae, protozoa, multi-cellular parasites, and viruses; tumor markers; liver function markers; kidney function markers; blood coagulation factors; and toxins. The embodiments may also optionally detect metabolites of each of the aforementioned examples of analytes. Furthermore, some embodiments may also detect their storage conditions, specifically the temperature and humidity of their environment, and/or the application of an appropriate quantity of sample for analysis.

Analytes may be reference analytes or target analytes. Any given analyte may be either a reference analyte or a target analyte, depending upon the desired application. Indeed, any analyte described below that is known to consistently be present in a given sample may serve as a reference analyte. As a non-limiting example, alpha-amylase is an enzyme present in saliva and could serve as a reference analyte when the analyzed sample is saliva. However, methadone could serve as a reference analyte when an embodiment is desired for use with samples obtained from patients generally known and/or suspected of having methadone in their system. Thus, one will readily appreciate that it is the application of the embodiment that determines the analytes classified as references or targets.

More specific examples of drug analytes, including both drugs of abuse and therapeutic drugs, include benzheterocyclics, the heterocyclic rings being azepines, diazepines and phenothiazines. Examples of azepines include fenoldopam. Examples of benzodiazepines include alprazolam, bretazenil, bromazepam, chlorodiazepoxide, cinolazepam, clonazepam, cloxazolam, clorazepate, diazepam, estazolam, fludiazepam, flunirazepam, flurazepam, flutoprazepam, halazepam, ketazolam, loprazolam, lorazepam, lormetazepam, medazepam, midazolam, nimetazepam, nitrazepam, nordazepam, oxazepam, phenazepam, pinazepam, prazepam, premazepam, quazepam, temazepam, tetrazepam, triazolam, and other benzodiazepine receptor ligands such as clobazam, DMCM, flumazenil, eszopiclone, zaleplon, zolpidem, and zopiclone. Examples of phenothiazines include chlorpromazine, promethazine, triflupromazine, methotrimeprazine, mesoridazine, thioridazine, fluphenazine, perphenazine, prochlorperazine, and trifluoperazine. Examples of other benzheterocyclics include, but are not limited to, carbamazepine and imipramine.

Additional drug analytes, including both drugs of abuse and therapeutic drugs, include alkaloids, such as agents that interact with opioid receptors including morphine, dihydromorphine, desomorphine, hydromorphone, nicomorphine, oxymorphone, hydromorphinol, nalbuphine, naloxone, naltrexone, buprenorphine, etorphine, metopon, diacetyldihydromorphine, thebacon, methodone, codeine, hydrocodone, dihydrocodeine, oxycodone, papaveretum, oripavine, thebaine, tapentadol, and heroin; agents that exert effects on serotonin receptors, such as cocaine (and other reuptake inhibitors, including norepinephrine, dopamine, and serotonin reuptake inhibitors); cocaine metabolites such as benzoylecgonine; ergot alkaloids; steroid alkaloids; iminazoyl alkaloids; quinazoline alkaloids; isoquinoline alkaloids; quinoline alkaloids; and diterpene alkaloids.

Another group of drug analytes, including both drugs of abuse and therapeutic drugs, includes steroids, including the estrogens, gestogens, androgens, andrenocortical steroids, bile acids, cardiotonic glycosides and aglycones, which includes digoxin and digoxigenin, saponins and sapogenins, their derivatives and metabolites.

Additional drug analytes, including both drugs of abuse and therapeutic drugs, is the barbiturates, such as barbital, allobarbital, amobarbital, aprobarbital, alphenal, brallobarbital, Phenobarbital, pentobarbital, Nembutal, secobarbital, diphenylhydantonin, primidone, and ethosuximide. Additionally, drugs similar in effect to barbiturates are potential analytes, such as methaqualone, cloroqualone, diproqualone, etaqualone, mebroqualone, mecloqualone, methylmethaqualone, and nitromethaqualone.

Another group of drug analytes, including both drugs of abuse and therapeutic drugs, is aminoalkylbenzenes, including the phenethylamines such as amphetamine, methamphetamine, lisdexamfetamine, mescaline, and catecholamines, which includes ephedrine, L-dopa, epinephrine, narceine, and papaverine.

Additional drug analytes, including both drugs of abuse and therapeutic drugs, includes those derived from marijuana, which includes cannabinol, tetrahydrocannabinol, 11-nor-9-carboxy-delta-9-tetrahydrocannabinol, nabilone, dronabinol, marinol, and cannabinoids such as cannabidiol, cannabinol, and tetrahydrocannabivarin.

Another group of drug analytes, including both drugs of abuse and therapeutic drugs, are those that interact with the N-methyl d-aspartate (“NMDA”) receptor, including agonists, modulators, and antagonists such as 1-(1-phylcyclohexyl)piperidine (phencyclidine or “PCP”), R-2-amino-5-phosphonopentanoate, 2-amino-7-phosphonoheptanoic acid, (3-[(R)-2-carboxypiperazin-4-yl]-prop-2-enyl-1-phosphonic acid), PEAQX, selfotel, amantadine, dextrallorphan, dextromethorphan, dextrorphan, dizocilpine, ethanol, eticyclidine, gacyclidine, ibogaine, ketamine, memantine, methoxetamine, rolicyclidine, tenocyclidine, tiletamine, neramexane, eliprodil, etoxadrol, dexoxadrol, NEFA, remacemide, delucemine, 8A-PDHQ, aptiganel, HU-211, remacemide, atomoxetine, rhynchophylline, 1-aminocyclopropanecarboxylic acid, 7-chlorokynurenate, 5,7-dichlorokynurenic acid, kynurenic acid, and lacosamide.

Another group of therapeutic drugs is antibiotics, which include, for example, beta-lactam antibiotics such as penicillins and cephalosporins, penems and carbapenems, antimicrobials such as aminoglycosides, ansamycins, carbacephems, glycopeptides, lincosamides, lipopetides, macrolides, monobactams, nitrofurans, quionolones, polypeptide-based antibiotics, chloromycetin, actinomycetin, spectinomycin, sulphonamides, trimethoprim, tetracyclines, and beta-lactamase inhibitors such as calvulanic acid, tazobactam, and sulbactam.

Other individual miscellaneous drug analytes, including both drugs of abuse and therapeutic drugs, include nicotine, caffeine, gamma-hydroxybutyric acid, dextromoramide, ketobemidone, piritramide, dipipanone, phenadoxone, benzylmorphine, nicocodeine, dihydrocodeinone enol acetate, tilidine, meptazinol, propiram, acetyldihydrocodeine, pholcodine, 3,4-methylenedioxymethamphetamine, psilocybin, 5-methoxy-N,N-diisopropyltryptamine, peyote, 2,5-dimethoxy-4-methylamphetamine, 2C-T-7 (a psychotropic entheogen), 2C-B, cathinone, alpha-methyltryptamine, bufotenin, benzylpiperazine, methylphenidate, dexmethylphenidate, laudanum, fentanyl, mixed amphetamine salts (i.e. Adderall), lisdexamfetamine, dextroamphetamine, dextromethamphetamine, pethidine, anabolic steroids, talbutal, butalbital, buprenorphine, xyrem, paregoric, modafinil, difenoxin, diphenoxylate, promethazine, pregabaline, pyrovalerone, atropine, and other Schedule I-V classified drugs, glucose, cholesterol, bile acids, fructosamine, carbohydrates, metals which includes, but is not limited to lead and arsenic, alcohols (i.e. methanol, ethanol, propanol, butanol, and C₅₋₁₀ containing alcohols), meprobamate, serotonin, meperidine, amitriptyline, nortriptyline, lidocaine, procaineamide, acetylprocainearnide, propranolol, griseofulvin, valproic acid, butyrophenones, antihistamines, and anticholinergic drugs, such as atropine.

Pesticide analytes of interest include categories such as algicides, avicides, bactericides, fungicides, herbicides, insecticides, miticides, molluscicides, nematicides, rodenticides, virucides, and specifically polyhalogenated biphenyls, phosphate esters, thiophosphates, carbamates, and polyhalogenated sulfenamides.

Additional chemical analytes of interest include fertilizers such as ammonium derivatives, nitrates, and phosphates; heavy metals such as lead, mercury, uranium, plutonium, arsenic, cadmium, chromium, and nickel

More specific examples of protein analytes include antibodies, protamines, histones, albumins, globulins, scleroproteins, phosphoproteins, mucoproteins, chromoproteins, lipoproteins, nucleoproteins, glycoproteins, proteoglycans, and unclassified proteins, such as somatotropin, prolactin, insulin, and pepsin. A number of proteins found in the human plasma are important clinically and include prealbumin, albumin, α₁-lipoprotein, α₁-acid glycoprotein, α₁-antitrypsin, α₁-glycoprotein, transcortin, 4.6S-postalbumin, tryptophan-poor, α₁-glycoprotein, α₁X-glycoprotein, thyroxin-binding globulin, inter-α-trypsin-inhibitor, Gc-globulin (Gc I-I, Gc 2-1, Gc 2-2), haptoglobin, ceruloplasmin, cholinesterase, α₂-lipoprotein(s), myoglobin, C-reactive Protein, α₂-macroglobulin, α₂-HS-glycoprotein, Zn-α₂-glycoprotein, α₂-neuramino-glycoprotein, erythropoietin, β-lipoprotein, transferrin, hemopexin, fibrinogen, plasminogen, β₂-glycoprotein I, β₂-glycoprotein II, immunoglobulins A, D, E, G, M, prothrombin, thrombin, and protein markers in cancers including, but not limited to, breast cancer, prostate cancer, melanoma, carcinoma, pancreatic cancer, liver cancer, and brain cancer.

Additional protein analytes of interest include alanine aminotransferase and aspartate aminotransferase. Alanine aminotransferase is markedly elevated when hepatitis is present in the liver. Such elevation for alanine aminotransferase may include at least about 1.25, 1.5, 1.75, 2, 2.25, 2.5, 2.75, and 3.0 times the normal levels associated with a person lacking liver damage. Aspartate aminotransferase is elevated when cellular damage occurs, such as liver damage, skeletal muscle damage, and acute myocardial infarction. Additionally, levels are elevated because of congestive heart failure, pericarditis, cirrhosis, metastatic liver disease, skeletal muscle diseases, and generalized infections such as mononucleosis. Such elevation for aspartate aminotransferase may include at least about 1.25, 1.5, 1.75, 2, 2.25, 2.5, 2.75, and 3.0 times the normal levels associated with a person lacking liver damage. Consequently, the detection of alanine aminotransferase and/or aspartate aminotransferase is of therapeutic importance.

Specific examples of peptide and protein hormone analytes include parathyroid hormone (parathromone), thyrocalcitonin, insulin, glucagon, relaxin, erythropoietin, melanotropin (melanocyte-stimulating hormone and intermedin), somatotropin (growth hormone), corticotropin (adrenocorticotropic hormone), thyrotropin, prolactin, follicle-stimulating hormone, luteinizing hormone), chorionic gonadotropin (hCG), oxytocin, and vasopressin.

Specific examples of polynucleotide analytes include DNA and RNA as well as their nucleoside and nucleotide precursors, which include ATP, NAD, FMN, adenosine, guanosine, thymidine, cytidine, and uracil with their appropriate sugar and phosphate substituents.

Specific examples of vitamin analytes include Vitamin A (i.e. retinol), B (e.g. B₁ or thiamine, B₂ or riboflavin, B₃ or niacin, B₅ or pantothenate, B₆ or pyridoxine, B₇ or biotin, B₉ or folic acid, and B₁₂), C (i.e. ascorbic acid), D (e.g. calciferol, D₂, and D₃), E (i.e. tocopherol), K, and vitamin derivatives or metabolites such as nicotinamide.

Specific examples of microorganism analytes, including infectious disease agents, include corynebacteria, pneumococci, streptococci, staphylococci, neisseriae, hemophilus influenzae, pasteurellae, brucellae, aerobic spore-forming bacilli, anaerobic spore-forming bacilli, mycobacteria, actinomycetes (fungus-like bacteria), the spirochetes, mycoplasmas, and other pathogens, such as listeria monocytogenes, erysipelothrix rhusiopathiae, streptobacillus moniliformis, donvania granulomatis, bartonella bacilliformis, rickettsiae (bacteria-like parasites), fungi, agents causing venereal diseases such as chlamydia, chancroid, granuloma inguinale, gonorrhea, syphilis, jock itch, yeast infection, herpes simplex, HPV, crab louse, scabies, trichomoniasis, and infectious diarrheal microorganisms such as camplylobacter, salmonellae, shigellae, Escherichia coli, Clostridium difficile, Giardia lamblia, Entamoeba histolytica, and organisms causing leptospirosis, nosocomial infections, staphylococcal enterotoxicosis, typhoid fever, cholera, vibrio gastroenteritis, yersinia gastroenteritis, clostridium perfringens gastroenteritis, bacillus cereus gastroenteritis, aflatoxin poisoning, amoebic dysentery, cryptosporidiosis, cyclospora diarrheal infection. Other microorganism analytes include viruses, such as herpes viruses, pox viruses, picornaviruses, myxoviruses (influenza A, B, and C, and mumps, measles, rubella, etc.), arboviruses, reoviruses, rotoviruses, noroviruses, adenoviruses, astroviruses, hepatitis, human immunodeficiency virus, and tumor viruses.

The categories of protein analytes and microorganism analytes may optionally overlap. For example, a microorganism analyte may be detected via the analysis of a protein analyte specific for the microorganism analyte. A protein analyte specific for a microorganism analyte may include an antibody specific for a microorganism analyte, or marker thereof. As a non-limiting example, for a microorganism analyte such as viral hepatitis, antibodies specific to any of viral hepatitis A, B, C, D, E, F and/or G may comprise the protein analyte. Such antibodies include, but are not limited to, immunoglobins such as IgA, IgD, IgE, and specifically IgM and/or IgG, and antibodies to surface antigens, envelope antigens, core antigens, and/or delta antigens (e.g. small and/or large). Specific examples of antigens for viral hepatitis B include hepatitis B surface antigen (HBsAg), hepatitis B envelope antigen (HBeAg), hepatitis B core antigen (HBcAg). Alternatively, a protein analyte specific for a microorganism analyte may include a protein analyte characteristically produced by the microorganism analyte. As a non-limiting example, for a microorganism analyte such as viral hepatitis, proteins specific to any of viral hepatitis A, B, C, D, E, and/or F may comprise the protein analyte. Such protein analytes include, but are not limited to, structural and/or nonstructural proteins. Specific examples of protein analytes for viral hepatitis C include, but are not limited to structural proteins such as E1 and/or E2, and/or nonstructural proteins such as NS2, NS3, NS4, NS4A, NS4B, NS5, NS5A, NS5B, and peptide portions thereof.

The above described analytes possess at least one marker recognized by at least one test reagent and/or signaling reagent. Optionally, the above described analytes may possess multiple markers recognized by the same and/or different test reagents and/or signaling reagents. It is readily envisioned that a marker may be the entire analyte and/or a portion thereof.

### Samples

An analyte of interest may be present in a wide variety of environments, and it is envisioned that a person having ordinary skill in the art will readily understand that the components and embodiments discussed above can be modified as needed to accommodate different environments of samples.

Analytes of interest may be found in a patient's physiological fluids, such as mucus, blood, serum, blood plasma, lymph, puss, urine, feces, cerebral spinal fluid, ocular lens liquid, ascites, semen, sputum, saliva, sweat, and secreted oils. Samples for testing analytes may be obtained using techniques known or envisioned to provide samples of such physiological fluids. Optionally, analytes may be detected by directly contacting embodiments of the diagnostic test strips with the patient's body, such as their skin, eyes, mouth cavity regions including the tongue, tonsils, and inner lining of the mouth and throat, and the nasal cavity. Alternatively, some analytes may be detected by directly contacting embodiments of the diagnostic test strips with a patient's urine stream, source of bleeding, source of puss, discharge from sex organs, or other site of fluid leakage from the patient.

Analytes may also be found in synthetic chemicals, water, soil, air and food (e.g., milk, meat, poultry, or fish). Any organic- and inorganic-containing substances can serve as an analyte so long as test reagents are available to generate a signal concerning the presence, absence, and/or concentration of the analyte.

For oral fluids such as saliva, samples may be obtained by contacting an embodiment with a patient's tongue such that the tongue contacts the one or more test pads. Alternatively, salivary samples may be obtained by contacting an embodiment with the top and/or sides of a patient's tongue using a substantially back and forth motion from substantially the tip of the tongue to substantially the back of the tongue. Furthermore, salivary samples may be obtained by contacting an embodiment with the top and/or sides of a patient's tongue using a substantially side-to-side motion along the width of the tongue. Similarly, salivary samples may also be obtained by contacting an embodiment with the top and/or sides of a patient's tongue using a substantially circular motion. For each of the above described sample collection methods, the results of the analysis could then be read directly from the diagnostic test strip by a user. Optionally, test results could be stored to a suitable memory device for recordation and later access.

Prior to use with embodiments of the invention, samples may be preserved, stored, or pre-treated in manners consistent with known handling of the same, or similar, types of samples. It is envisioned that any type of preservation, storage, or pre-treatment may be utilized so long as it does not introduce false positives or false negatives into the assay.

### CONCLUSION

While the invention has been described with reference to the specific embodiments thereof, it should be understood by those skilled in the art that various changes may be made and equivalents may be substituted without departing from the true spirit and scope of the invention. This includes embodiments which do not provide all of the benefits and features set forth herein. In addition, many modifications may be made to adapt a particular situation, material, composition of matter, process, process step or steps, to the objective, spirit and scope of the present invention. All such modifications are intended to be within the scope of the claims appended hereto. Accordingly, the scope of the invention is defined only by reference to the appended claims.

What is claimed is:

1. An apparatus that is a diagnostic test strip comprising
a) a carrier strip with one or more sets of perforations;
b) one or more test pads with a plurality of legs that are attached to and extend away from the pad,
wherein the sets of perforations are configured to accept the plurality of legs on each of the test pads.

2. The diagnostic test strip of claim 1, wherein the legs of the test pads extend at least partially into the carrier strip.

3. The diagnostic test strip of claim 1, wherein the legs of the test pads extend through and past the opposing side of the carrier strip.

4. The diagnostic test strip of claim 1, wherein the legs of the test pads extend through and past the opposing side of the carrier strip and the portion of the legs that extend past the opposing side are bent to the surface of the opposing side of the carrier strip.

5. The diagnostic test strip of claim 1, wherein the legs of the test pads extend through and past the opposing side of the carrier strip and the portion of the legs that extend past the opposing side are bent to the surface of, and a portion of leg is further inserted into, the opposing side of the carrier strip.

6. The diagnostic test strip of claim 1, wherein at least one of the test pads contains a test reagent.

7. The diagnostic test strip of claim 1, wherein there are more than one test pad and each one contains a different test reagent.

8. The diagnostic test strip of claim 1, wherein the test pads are substantially square.

9. The diagnostic test strip of claim 1, wherein the test pads are substantially circular.

10. The diagnostic test strip of claim 1, wherein each test pad has at least one or more legs.

11. The diagnostic test strip of claim 1, wherein each test pad has at least two legs.

12. The diagnostic test strip of claim 1, wherein each test pad has at least three legs.

13. The diagnostic test strip of claim 1, wherein each test pad has four legs.

14. The diagnostic test strip of claim 1, wherein the carrier strip is substantially porous.

15. The diagnostic test strip of claim 1, wherein the carrier strip is substantially non-porous.

16. The diagnostic test strip of claim 1, wherein the test pads are substantially porous.

17. The diagnostic test strip of claim 1, wherein the test pads are substantially non-porous.

18. The diagnostic test strip of claim 1, wherein the test pads extend substantially the entire width of the carrier strip.

19. The diagnostic strip of claim 1, wherein at least one test pad is substantially at one end of the carrier strip.

20. The diagnostic strip of claim 1, wherein a plurality of test pads are placed sequentially over the length of the carrier strip with a defined area separating each test pad on the carrier strip.

21. The diagnostic test strip of claim 1, wherein there are at least two or more test pads each with a different test reagent and each reagent tests for a different marker on the same analyte.

22. The diagnostic test strip of claim 1, wherein at least one test pad further contains a signaling reagent.

23. The diagnostic test strip of claim 1, wherein the at least one test pad contains a reagent that tests for a saliva-borne analyte.

24. The diagnostic test strip of claim 1, wherein the at least one test pad contains a reagent that tests for a sputum-borne analyte.

25. The diagnostic test strip of claim 1, wherein the at least one test pad contains a reagent that tests for a serum-borne analyte.

26. The diagnostic test strip of claim 1, wherein the at least one test pad contains a reagent that tests for a plasma-borne analyte.

27. The diagnostic test strip of claim 1, wherein the at least one test pad contains a reagent that tests for a blood-borne analyte.

28. The diagnostic test strip of claim 1, wherein the at least one test pad contains a reagent that tests for a urine-borne analyte.

29. The diagnostic test strip of claim 1, wherein the at least one test pad contains a reagent that tests for a semen-borne analyte.

30. The diagnostic test strip of claim 1, wherein the at least one test pad contains a reagent that tests for an ascites-borne analyte.

31. The diagnostic test strip of claim 1, wherein the at least one test pad contains a reagent that tests for a cerebral spinal fluid-borne analyte.

32. A method for detecting one or more analytes in a patient sample, comprising:
a) contacting the test strip of claim 1 with a patient sample so that the sample contacts the one or more test pads; and
b) reading the results from the test strip.

33. The method of claim 32, further comprising contacting the test strip with one or more signaling reagents so that the one or more signaling reagents contact the one or more test pads.

34. The method of claim 32, wherein the patient sample is serum.

35. The method of claim 32, wherein the patient sample is semen.

36. The method of claim 32, wherein the patient sample is urine.

37. The method of claim 36, wherein the test strip is directly contacted with a patient's urine stream.

38. The method of claim 32, wherein the patient sample is saliva.

39. The method of claim 38, wherein the test strip is contacted with a patient's tongue.

40. The method of claim 32, wherein the patient sample is blood.

41. The method of claim 40, wherein the test strip is contacted directly with a source of the blood.

42. The method of claim 32, wherein the patient sample is ascites.

43. The method of claim 32, wherein the patient sample is sputum.

44. The method of claim 32, wherein the patient sample is cerebral spinal fluid.

45. The diagnostic test strip of claim 1, wherein the one or more test pads further comprises:
a) a first transparent membrane containing a test reagent that indicates the presence of at least one reference analyte; and
b) a second transparent membrane containing a test reagent that indicates the presence of at least one target analyte;
wherein each of the test reagents are arranged in a substantially single striped shape on a portion of the transparent membranes, and the transparent membranes are opposed to each other such that the striped shapes are at substantially right angles, and the at least one test pad is in fluid contact with the diagnostic test strip.
